(19) United States
(12) Patent Application Publication
(10) Pub. No.: US 2019/0209137 A1
(43) Pub. Date: Jul. 11, 2019

(54) INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventors: Taku Inoue, Machida-shi (JP); Kazuhito Oka, Tokyo (JP)

(21) Appl. No.: 16/353,537

(22) Filed: Mar. 14, 2019

Related U.S. Application Data
(63) Continuation of application No. PCT/JP2017/041317, filed on Nov. 16, 2017.

(30) Foreign Application Priority Data
Nov. 24, 2016 (JP) 2016-228065

(51) Int. Cl. A61B 8/08; A61B 5/00; A61B 8/14

(52) U.S. Cl. CPC A61B 8/5207; A61B 8/14; A61B 5/0095; A61B 5/0059

(57) ABSTRACT

An information processing apparatus obtains an ultrasonic image at a frame rate higher than that of a photoacoustic image. A first photoacoustic image is obtained at a first time point, a second photoacoustic image is obtained at a second time point after the first time point, and a first ultrasonic image is obtained at a third time point included in a period of time after the first time point and before the second time point. At least one of the first and second photoacoustic images is associated with the first ultrasonic image based on the relationship among the first to third time points.

### CROSS-REFERENCE TO RELATED APPLICATIONS

This application is a Continuation of International Patent Application No. PCT/JP2017/041317, filed Nov. 16, 2017, which claims the benefit of Japanese Patent Application No. 2016-228065, filed Nov. 24, 2016, both of which are hereby incorporated by reference herein in their entirety.

### TECHNICAL FIELD

The present invention relates to an information processing apparatus, an information processing method, and a storage medium.

### BACKGROUND ART

As an imaging apparatus which generates a minimally invasive image of an internal state of a subject, ultrasonic imaging apparatuses and photoacoustic imaging apparatuses have been used. According to PTL 1, an apparatus capable of obtaining an ultrasonic image in a frame rate higher than that of a photoacoustic image generates supplementary information including information on a frame of a photoacoustic image corresponding to a frame of an ultrasonic image and information indicating that a frame of a photoacoustic image corresponding to a frame of an ultrasonic image does not exist.

### CITATION LIST

### Patent Literature

PTL 1 Japanese Patent Laid-Open No. 2014-217652

When a moving image including an ultrasonic image and a photoacoustic image is reproduced based on supplementary information including information on a frame of the photoacoustic image corresponding to a frame of the ultrasonic image and information indicating that a frame of the photoacoustic image corresponding to a frame of the ultrasonic image does not exist, a time point when the photoacoustic image is displayed and a time point when the photoacoustic image is not displayed are mixed, and accordingly, such a moving image may be hard to be viewed by a user.

### SUMMARY OF INVENTION

An information processing apparatus according to an embodiment of the present invention includes signal obtaining means which obtains a first photoacoustic signal which associates with a photoacoustic wave generated due to emission of light to a subject at a first time point and a second photoacoustic signal at a second time point after the first time point and which obtains an ultrasonic signal which is associated with a reflection wave of an ultrasonic wave emitted to the subject at a third time point included in a period of time between the first and second time points, image obtaining means for obtaining an ultrasonic image based on the ultrasonic signal and a photoacoustic image based on the photoacoustic signal, and processing means for associating one of the generated photoacoustic images with the ultrasonic image generated based on the ultrasonic signal obtained at the third time point based on the first and second photoacoustic signals.

Further features of the present invention will become apparent from the following description of exemplary embodiments with reference to the attached drawings.

### BRIEF DESCRIPTION OF DRAWINGS

FIG. 1 is a diagram illustrating an example of a configuration of a system including an information processing apparatus according to an embodiment of the present invention.

FIG. 2 is a diagram illustrating an example of a hardware configuration of the information processing apparatus according to the embodiment of the present invention.

FIG. 3 is a diagram illustrating an example of a functional configuration of the information processing apparatus according to the embodiment of the present invention.

FIG. 4 is a flowchart of an example of a process performed by the information processing apparatus according to the embodiment of the present invention.

FIG. 5 is a diagram illustrating an example of a configuration of data generated by the information processing apparatus according to the embodiment of the present invention.

FIG. 6 is a diagram illustrating an example of supplementary information generated by the information processing apparatus according to the embodiment of the present invention.

FIG. 7 is a diagram illustrating an example of a configuration of an object output to an external apparatus from the information processing apparatus according to the embodiment of the present invention.

FIG. 8 includes timing charts of an example of a process performed by the information processing apparatus according to the embodiment of the present invention.

FIG. 9 is a flowchart of an example of a process performed by the information processing apparatus according to the embodiment of the present invention.

FIG. 10 is a flowchart of an example of a process performed by an information processing apparatus according to an embodiment of the present invention.

FIG. 11 is a diagram illustrating an example of data generated by the information processing apparatus according to the embodiment of the present invention.

FIG. 12 is a diagram illustrating an example of supplementary information generated by the information processing apparatus according to the embodiment of the present invention.

FIG. 13 is a diagram illustrating an example of a configuration of an object output to an external apparatus from the information processing apparatus according to the embodiment of the present invention.

FIG. 14 includes timing charts of an example of a process performed by the information processing apparatus according to the embodiment of the present invention.

FIG. 15 is a flowchart of an example of a process performed by the information processing apparatus according to the embodiment of the present invention.

FIG. 16 is a flowchart of an example of a process performed by an information processing apparatus according to an embodiment of the present invention.

FIG. 17 is a diagram illustrating an example of supplementary information generated by the information processing apparatus according to the embodiment of the present invention.

FIG. 18 is a diagram illustrating an example of supplementary information generated by the information processing apparatus according to the embodiment of the present invention.

### DESCRIPTION OF EMBODIMENTS

Hereinafter, embodiments of the present invention will be described with reference to the accompanying drawings.

### First Embodiment

In this specification, an acoustic wave generated by expansion generated inside a subject when the subject is irradiated with light is referred to as a “photoacoustic wave”. Furthermore, an acoustic wave transmitted from a transducer or a reflection wave (an echo) obtained by reflecting the acoustic wave transmitted from the transducer in an inside of a subject is referred to as an “ultrasonic wave”.

As a method for generating a minimally invasive image of an internal state of a subject, a method for generating an image using an ultrasonic wave or a method for generating an image using a photoacoustic wave has been used. In the method for generating an image using an ultrasonic wave, an ultrasonic wave which is oscillated by a transducer, for example, is reflected by a tissue in a subject in accordance with a difference of acoustic impedance, and an image is generated based on a period of time until a reflection wave reaches the transducer and intensity of the reflection wave. An image generated using an ultrasonic wave is referred to as an “ultrasonic image” hereinafter. A user operates a probe while changing an angle or the like of the probe so as to observe ultrasonic images of various cross-sectional views in real time. The ultrasonic image includes shapes of organs and tissues rendered therein which are used to find a tumor or the like. Furthermore, in the method for generating an image using a photoacoustic wave, an image is generated based on an ultrasonic wave (a photoacoustic wave) generated when a tissue in a subject which has been irradiated with light is adiabatically expanded. An image generated using a photoacoustic wave is referred to as a “photoacoustic image” hereinafter. In the photoacoustic image, information on optical characteristics including a degree of light absorption of tissues is rendered. In the photoacoustic image, blood vessels may be rendered by optical characteristics of hemoglobin, and therefore, use of a photoacoustic image in evaluation of a degree of malignancy of a tumor and the like has been discussed.

To enhance accuracy of diagnosis, various information may be collected by generating images of different phenomena based on different principles in the same portion of a subject. An imaging apparatus which performs imaging of an ultrasonic image and imaging of a photoacoustic image and which obtains an image by combining characteristics of the ultrasonic image and the photoacoustic image has been discussed. In particular, an ultrasonic image and a photoacoustic image are similarly generated using ultrasonic waves transmitted from a subject, and therefore, imaging of an ultrasonic image and imaging of a photoacoustic image may be performed by the same imaging apparatus. Specifically, a reflection wave and a photoacoustic wave emitted to the subject may be received by the same transducer. By this, an ultrasonic signal and a photoacoustic signal may be obtained by a single probe, and therefore, an imaging apparatus which performs imaging of an ultrasonic image and imaging of a photoacoustic image may be realized without a complicated hardware configuration.

In this way, in a case where a still image or a moving image is captured using an imaging apparatus capable of obtaining an ultrasonic image and a photoacoustic image, the user may observe an ultrasonic image and a photoacoustic image while comparing the images. Therefore, an ultrasonic image and a photoacoustic image which are to be compared with each other are preferably associated with each other in advance in terms of a workflow when the user observes the images. However, a process of generating a photoacoustic image by obtaining a photoacoustic signal may require a longer period of time when compared with a process of generating an ultrasonic image by obtaining an ultrasonic signal. In this case, when a moving image constituted by an ultrasonic image and a photoacoustic image is captured, a frame rate of the photoacoustic image may be lower than that of the ultrasonic image. Therefore, a portion of the ultrasonic image of a high frame rate is not associated with the photoacoustic image only by associating the ultrasonic image and the photoacoustic image which are obtained based on an ultrasonic signal and a photoacoustic signal which are obtained substantially at the same time with each other. If the user reproduces such a moving image using a viewer, a photoacoustic image is intermittently displayed in a moving image of an ultrasonic image, and therefore, visibility of the moving image may be degraded. A first embodiment is made to output data in which an ultrasonic image and a photoacoustic image are smoothly reproduced when a moving image is reproduced by a viewer.

Configuration of Information Processing Apparatus

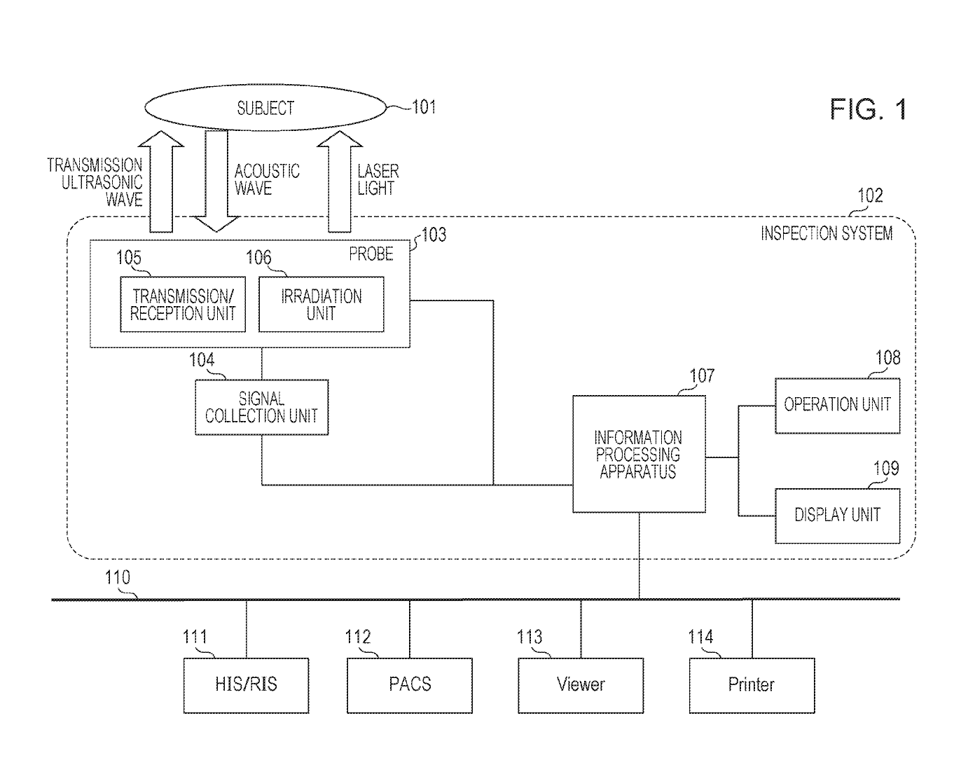

FIG. 1 is a diagram illustrating an example of a configuration of an inspection system 102 including an information processing apparatus 107 according to the first embodiment. The inspection system 102 capable of generating an ultrasonic image and a photoacoustic image is connected to various types of external apparatus through a network 110. Various components included in the inspection system 102 and the various external apparatuses are not required to be installed in the same facility as long as the components and the external apparatuses are connected to one another in a communication available manner.

The inspection system 102 includes the information processing apparatus 107, a probe 103, a signal collection unit 104, a display unit 109, and an operation unit 108. The information processing apparatus 107 obtains information on inspection including imaging of an ultrasonic image and a photoacoustic image from an HIS/RIS 111 and controls the probe 103 and the display unit 109 when the inspection is performed. The information processing apparatus 107 obtains an ultrasonic signal and a photoacoustic signal from the probe 103 and the signal collection unit 104. The information processing apparatus 107 obtains an ultrasonic image based on the ultrasonic signal and a photoacoustic image based on the photoacoustic signal. The information processing apparatus 107 may further obtain a superposed image by superposing a photoacoustic image on an ultrasonic image. The information processing apparatus 107 performs transmission and reception of information with an external apparatus, such as the HIS/RIS 111 or a PACS 112, based on a standard of Health level 7 (HL7) or Digital Imaging and Communications in Medicine (DICOM).

Examples of a region in a subject 101 in which an ultrasonic image is captured by the inspection system 102 include a circulatory organ region, a breast, a liver, a pancreas, and an abdomen. Furthermore, the inspection system 102 may capture an ultrasonic image of a subject to which an ultrasonic contrast agent utilizing microbubbles is administered.

Furthermore, examples of a region in a subject subjected to imaging so that a photoacoustic image is obtained by the inspection system 102 include a circulatory organ region, a breast, an inguinal area, an abdomen, and four extremities including fingers and toes. In particular, a blood vessel region including a new blood vessel and plaque of a blood vessel wall may be a target of imaging of a photoacoustic image in accordance with characteristics of light absorption in the subject. The inspection system 102 may capture a photoacoustic image of the subject 101 to which a substance obtained by accumulating or chemically modifying pigments, such as methylene blue or indocyaninegreen, or gold fine particles is administered as a contrast agent.

The probe 103 is operated by the user so as to transmit an ultrasonic signal and a photoacoustic signal to the signal collection unit 104 and the information processing apparatus 107. The probe 103 includes a transmission/reception unit 105 and an irradiation unit 106. The probe 103 transmits an ultrasonic wave from the transmission/reception unit 105 and receives a reflection wave by the transmission/reception unit 105. Furthermore, the probe 103 causes the irradiation unit 106 to irradiate the subject with light and causes the transmission/reception unit 105 to receive a photoacoustic wave. When receiving information on contact with the subject, the probe 103 is preferably controlled such that transmission of an ultrasonic wave for obtaining an ultrasonic signal and light irradiation for obtaining a photoacoustic signal are executed.

The transmission/reception unit 105 includes at least one transducer (not illustrated), a matching layer (not illustrated), a damper (not illustrated), and a photoacoustic lens (not illustrated). The transducer (not illustrated) is constituted by a substance having a piezoelectric effect, such as a lead zirconate titanate (PZT) or polyvinylidene difluoride (PVDF). The transducer (not illustrated) may not be a piezoelectoric element, and may be a capacitive micro-machined ultrasonic transducer (CMUT) or a transducer using a Fabry-Perot interferometer, for example. Typically, an ultrasonic signal has a frequency component in a range from 2 to 20 MHz and a photoacoustic signal has a frequency component in a range from 0.1 to 100 MHz, and the transducer (not illustrated) is capable of detecting the frequencies. A signal obtained by the transducer (not illustrated) is a time-resolved signal. Amplitude of the received signal indicates a value based on a sound pressure received by the transducer at each time point. The transmission/reception unit 105 includes a circuit (not illustrated) for electronic focus or a controller. An arrangement shape of transducers (not illustrated) is a sector, linear array, convex, annular array, or matrix array, for example. The probe 103 obtains an ultrasonic signal and a photoacoustic signal. The probe 103 obtains an ultrasonic signal and a photoacoustic signal in an alternate manner, a simultaneous manner, or a predetermined mode.

The transmission/reception unit 105 may include an amplifier (not illustrated) which amplifies analog signals which are received in time-series by the transducer (not illustrated). The transducers (not illustrated) may be divided into those for transmission and those for reception in accordance with a purpose of imaging of an ultrasonic image. Furthermore, the transducers (not illustrated) may be divided into those for imaging of an ultrasonic image and those for imaging of a photoacoustic image.

The irradiation unit 106 includes a light source (not illustrated) which obtains a photoacoustic signal and an optical system (not illustrated) which guides pulse light emitted from the light source (not illustrated) to the subject. The light emitted from the light source (not illustrated) has a pulse width in a range from 1 ns inclusive to 100 ns inclusive. Furthermore, the light emitted from the light source (not illustrated) has a wavelength in a range from 400 nm inclusive to 1600 nm inclusive. When a blood vessel in the vicinity of a surface of the subject is to be imaged in high resolution, a wavelength of high absorption in blood vessels which is in a range from 400 nm inclusive to 700 nm inclusive is preferably used. Furthermore, when a depth portion of the subject is to be imaged, a wavelength in a range from 700 nm inclusive to 1100 nm inclusive which is difficult to be absorbed by water or tissues, such as fat, is preferably used.

The light source (not illustrated) is a laser or a light emitting diode, for example. The irradiation unit 106 may be a light source which may convert a wavelength so as to obtain a photoacoustic signal using light of a plurality of wavelengths. Alternatively, the irradiation unit 106 may include a plurality of light sources which generate different light beams of different wavelengths, and the different light sources may alternately emit light of different wavelengths. Examples of the laser include a solid-state laser, a gas laser, a dye laser, and a semiconductor laser. A pulse laser, such as an Nd:YAG laser or an alexandrite laser, may be used as the light source (not illustrated). A Ti:sa laser which uses light of an Nd:YAG laser as excitation light or an optical parametric oscillators (OPO) laser may be used as the light source (not illustrated). Furthermore, as the light source (not illustrated), a microwave source may be used.

An optical element, such as a lens, a mirror, or an optical fiber, is used for the optical system (not illustrated). When the subject is a breast, it is preferable that a beam diameter of the pulse light is increased before emission, and therefore, the optical system (not illustrated) may include a diffusion plate which diffuses light emitted from the optical system (not illustrated). Alternatively, the optical system (not illustrated) may include a lens and the like so as to focus a beam so that high resolution is attained.

The signal collection unit 104 converts analog signals of the reflection wave and the photoacoustic wave received by the probe 103 into digital signals. The signal collection unit 104 transmits the ultrasonic signal and the photoacoustic signal which have been converted into the digital signals to the information processing apparatus 107.

The display unit 109 displays an image captured by the inspection system 102 and information associated with the inspection under control of the information processing apparatus 107. The display unit 109 is provided with interfaces which receive instructions issued by the user under control of the information processing apparatus 107. The display unit 109 is a liquid crystal display, for example.

The operation unit 108 transmits information on an operation input performed by the user to the information processing apparatus 107. The operation unit 108 includes a keyboard, a trackball, and various buttons for inputting operations associated with the inspection.

Note that the display unit 109 and the operation unit 108 may be integrated as a touch panel display. Furthermore, the information processing apparatus 107, the display unit 109, and the operation unit 108 may not be different devices and may be realized as an integrated operation console. The information processing apparatus 107 may include a plurality of probes.

The HIS/RIS 111 is a system which manages information on a patient and information on inspection. The hospital information system (HIS) assists services in hospitals. The HIS includes an electronic medical record system, an ordering system, and a medical business accounting system. The radiology information system (RIS) manages inspection information in a radiology department and manages progress of inspection in the imaging apparatus. The inspection information includes an inspection ID for unique identification and information on an imaging technique included in the inspection. The inspection system 102 may be connected to ordering systems constituted for individual departments instead of the RIS or in addition to the RIS. The HIS/RIS 111 manages an order issuance of the inspection to accounting in cooperation. The HIS/RIS 111 transmits the information on the inspection performed by the inspection system 102 to the information processing apparatus 107 in accordance with an inquiry supplied from the information processing apparatus 107. The HIS/RIS 111 receives information on progress of the inspection from the information processing apparatus 107. When receiving information on completion of the inspection from the information processing apparatus 107, the HIS/RIS 111 performs an accounting process.

A picture archiving and communication system (PACS) 112 is a database system which stores images obtained by various types of imaging apparatus in and out of the facility. The PACS 112 includes a storage unit (not illustrated) which stores medical images, imaging conditions of the medical images, parameters of image processing including a reconfiguration, and supplementary information, such as patient information and a controller (not illustrated) which manages information stored in the storage unit. The PACS 112 stores the ultrasonic image, the photoacoustic image, and the superposed image which are objects output from the information processing apparatus 107. The Communication between the PACS 112 and the information processing apparatus 107 and various images stored in the PACS 112 are preferably based on a certain standard, such as the HL7 or the DICOM. The various images output from the information processing apparatus 107 have various tags associated with supplementary information based on the DICOM standard when being stored.

A viewer 113 which is an image diagnosis terminal reads images stored in the PACS 112 and the like and performs display for diagnosis. A doctor observes an image displayed in the viewer 113 and records information obtained as a result of the observation as an image diagnosis report. The image diagnosis report generated by the viewer 113 may be stored in the viewer 113 or output to the PACS 112 or a report server (not illustrated) which stores the image diagnosis report.

A printer 114 prints an image stored in the PACS 112 or the like. The printer 114 is a film printer, for example, which performs output by printing an image stored in the PACS 112 or the like in a film.

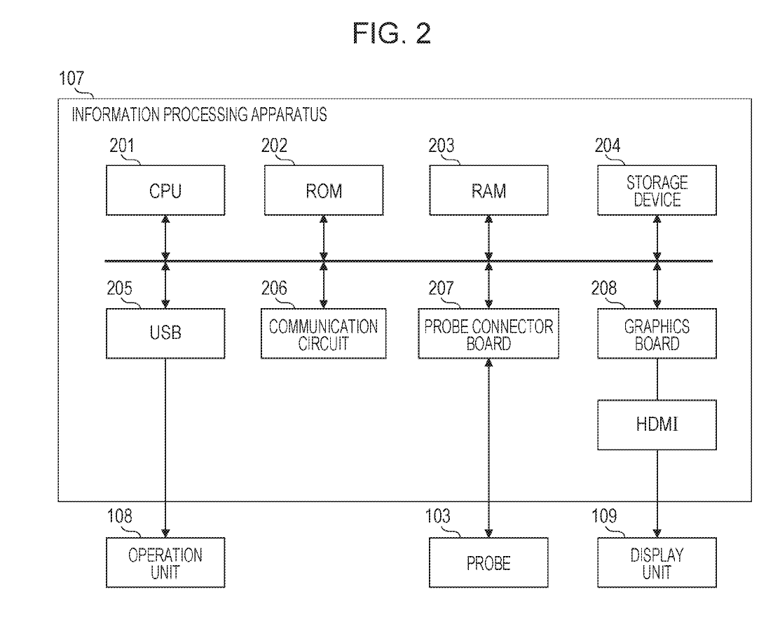

FIG. 2 is a diagram illustrating an example of a hardware configuration of the information processing apparatus 107. The information processing apparatus 107 is a computer, for example. The information processing apparatus 107 includes a central processing unit (CPU) 201, a read only memory (ROM) 202, a random access memory (RAM) 203, a storage device 204, a universal serial bus (USB) 205, a communication circuit 206, a probe connector port 207, and a graphics board 208. The devices are connected to one another through a bus in a communication available manner. The bus is used for transmission and reception of data between hardware portions connected to each other and transmission of an instruction from the CPU 201 to the other hardware portions.

The central processing unit (CPU) 201 is a control circuit which integrally controls the information processing apparatus 107 and the components connected to the information processing apparatus 107. The CPU 201 performs control by executing programs stored in the ROM 202. Furthermore, the CPU 201 executes a display driver which is software for controlling the display unit 109 and performs display control on the display unit 109. Furthermore, the CPU 201 performs input/output control on the operation unit 108.

The read only memory (ROM) 202 stores programs and data storing a procedure of control by the CPU 201. The ROM 202 stores a boot program and various initial data of the information processing apparatus 107. The ROM 202 further stores various programs for realizing processes performed by the information processing apparatus 107.

The random access memory (RAM) 203 is used as a storage region for operations when the CPU 201 performs control in accordance with an instruction program. The RAM 203 includes a stack area and a work area. The RAM 203 stores programs used when the information processing apparatus 107 and the components connected to the information processing apparatus 107 perform processes and various parameters used in the image processing. The RAM 203 stores a control program to be executed by the CPU 201 and temporarily stores various data used when the CPU 201 executes various control operations.

The storage device 204 is an auxiliary storage device which stores various data including an ultrasonic image and a photoacoustic image. The storage device 204 is a hard disk drive (HDD) or a solid state drive (SSD), for example.

The universal serial bus (USB) 205 is a connection unit connected to the operation unit 108.

The communication circuit 206 performs communication with the units included in the inspection system 102 and the various external apparatuses connected to the network 110. The communication circuit 206 stores information to be output in a transfer packet and outputs the packet to an external apparatus through the network 110 by a communication technique, such as TCP/IP. The information processing apparatus 107 may include a plurality of communication circuits in accordance with a desired communication form.

The probe connector port 207 is a connection port used to connect the probe 103 to the information processing apparatus 107.

The graphics board 208 includes a graphics processing unit (GPU) and a video memory. The GPU performs calculation associated with a reconfiguration process of generating a photoacoustic image using a photoacoustic signal, for example.

A high definition multimedia interface (HDMI: registered trademark) 209 is a connection unit connected to the display unit 109.

The CPU 201 and the GPU are examples of a processor. Furthermore, the ROM 202, the RAM 203, and the storage device 204 are examples of a memory. The information processing apparatus 107 may include a plurality of processors. In the first embodiment, functions of the units included in the information processing apparatus 107 are realized when the processor of the information processing apparatus 107 executes the programs stored in the memory.

Furthermore, the information processing apparatus 107 may include a CPU, a GPU, and an application specific integrated circuit (ASIC) which dedicatedly perform specific processes. The information processing apparatus 107 may include a field-programmable gate array (FPGA) in which a specific process or all processes are programmed.

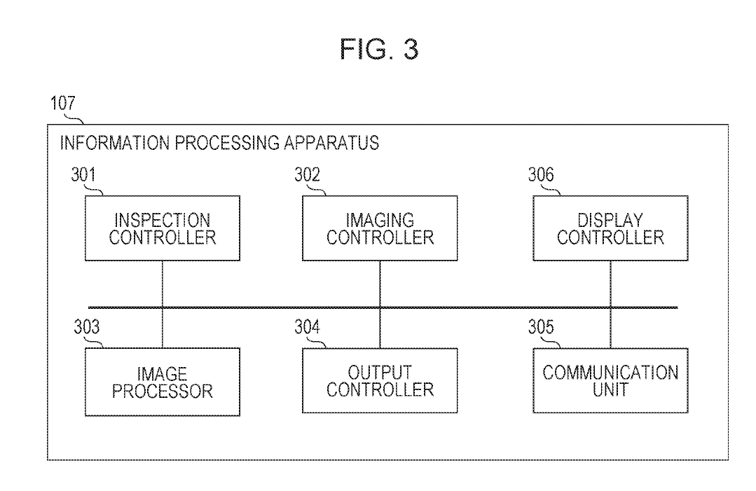

FIG. 3 is a diagram illustrating an example of a functional configuration of the information processing apparatus 107. The information processing apparatus 107 includes an inspection controller 301, an imaging controller 302, an image processor 303, an output controller 304, a communication unit 305, and a display controller 306.

The inspection controller 301 obtains information on an inspection order from the HIS/RIS 111. The inspection order includes information on a patient who is to be inspected and information on an imaging technique. The inspection controller 301 transmits information on the inspection order to the imaging controller 302.

Furthermore, the inspection controller 301 causes the display unit 109 to display the information on inspection so as to display the information on inspection for the user through the display controller 306. The information on inspection displayed in the display unit 109 includes the information on a patient who is to be inspected, the information on an imaging technique included in the inspection, and an image generated after imaging is completed. Furthermore, the inspection controller 301 transmits information on progress of the inspection to the HIS/RIS 111 through the communication unit 305.

The imaging controller 302 controls the probe 103 based on the information on an imaging technique received from the inspection controller 301 so as to obtain an ultrasonic signal and a photoacoustic signal from the probe 103 and the signal collection unit 104. The imaging controller 302 instructs the irradiation unit 106 to emit light. The imaging controller 302 instructs the transmission/reception unit 105 to transmit an ultrasonic wave. The imaging controller 302 issues instructions to the irradiation unit 106 and the transmission/reception unit 105 by an operation input performed by the user or the information on an imaging technique. Furthermore, the imaging controller 302 instructs the transmission/reception unit 105 to receive the ultrasonic wave. The imaging controller 302 instructs the signal collection unit 104 to perform signal sampling. The imaging controller 302 controls the probe 103 as described above so as to obtain an ultrasonic signal and a photoacoustic signal in a discriminated manner. Furthermore, the imaging controller 302 obtains information on a timing when the ultrasonic signal is obtained and a timing when the photoacoustic signal is obtained (hereinafter referred to as “timing information”). The timing information indicates a timing when the imaging controller 302 controls the probe 103 so that light is emitted or an ultrasonic wave is transmitted. The information indicating a timing may be a time point or an elapsed period of time after start of the inspection. Note that the imaging controller 302 obtains an ultrasonic signal and a photoacoustic signal which have been converted into digital signals and which have been output from the signal collection unit 104. Specifically, the imaging controller 302 is an example of signal obtaining means which obtains an ultrasonic signal and a photoacoustic signal. The imaging controller 302 is also an example of information obtaining means which obtains timing information.

The image processor 303 generates an ultrasonic image, a photoacoustic image, and a superposed image obtained by superposing the photoacoustic image on the ultrasonic image. Furthermore, the image processor 303 generates a moving image including the ultrasonic image and the photoacoustic image.

Specifically, the image processor 303 generates a photoacoustic image based on the photoacoustic signal obtained by the imaging controller 302. The image processor 303 reconfigures distribution of acoustic waves obtained when light is emitted based on the photoacoustic signal (hereinafter referred to as an initial sound pressure distribution). The image processor 303 obtains a light absorption coefficient distribution in the subject by dividing the reconfigured initial sound pressure distribution by a light fluence distribution of the light emitted to the subject. Furthermore, the image processor 303 obtains a density distribution of substances in the subject in accordance with the absorption coefficient distribution relative to a plurality of wavelengths utilizing a difference between degrees of light absorption in the subject in accordance with a wavelength of the light emitted to the subject. For example, the image processor 303 obtains substance density distributions of oxyhemoglobin and deoxyhemoglobin in the subject. Furthermore, the image processor 303 obtains an oxygen saturation distribution as a rate of a density of oxyhemoglobin to a density of deoxyhemoglobin. The photoacoustic image generated by the image processor 303 indicates information on at least one of the initial sound pressure distribution, the light fluence distribution, the absorption coefficient distribution, the substance density distribution, and the oxygen saturation distribution described above, for example.

Furthermore, the image processor 303 obtains an emission line in which amplitude of a reflection wave of the ultrasonic signal is converted into luminance and generates an ultrasonic image (a B mode image) by changing a display position of the emission line in accordance with scanning using an ultrasonic beam. When the probe 103 is a 3D probe, the image processor 303 may generate an ultrasonic image (a C mode image) having three cross-sections which are orthogonal to one another. The image processor 303 generates an arbitrary cross-section or a 3D image after rendering based on the 3D ultrasonic image. The image processor 303 is an example of image obtaining means for obtaining an ultrasonic image and a photoacoustic image.

The output controller 304 generates an object for transmitting various information to an external apparatus, such as the PACS 112 or the viewer 113, in accordance with control performed by the inspection controller 301 or an operation input performed by the user. The object is information to be transmitted to the external apparatus, such as the PACS 112 or the viewer 113, from the information processing apparatus 107. For example, the output controller 304 generates a DICOM object for outputting the ultrasonic image, the photoacoustic image, and the superposed image which are generated by the image processor 303 to the PACS 112. The object output to the external apparatus includes supplementary information attached as various tags based on the DICOM standard. The supplementary information includes, for example, patient information, information on an imaging apparatus which has captured the image, an image ID for uniquely identifying the image, an inspection ID for uniquely identifying inspection in which the image has been captured, and information on the probe 103.

Furthermore, the supplementary information generated by the output controller 304 includes information on association between the ultrasonic image and the photoacoustic image which have been captured in the inspection. In a moving image including ultrasonic images and photoacoustic images, the ultrasonic images and the photoacoustic images may be obtained in different frame rates. For example, in a case where ultrasonic images are obtained in a higher frame rate, it is not necessarily the case that all the ultrasonic images have been obtained at substantially the same timings as corresponding photoacoustic images. The output controller 304 associates the plurality of ultrasonic images included in the moving image with the respective photoacoustic images based on the timing information obtained by the imaging controller 302. Then information indicating the photoacoustic images corresponding to the ultrasonic images is generated as supplementary information. The output controller 304 is an example of processing means which associates an ultrasonic image with one of photoacoustic images.

The communication unit 305 controls transmission and reception of information between the external apparatus, such as the HIS/RIS 111, the PACS 112, or the viewer 113 and the information processing apparatus 107 through the network 110. The communication unit 305 receives information on the inspection order from the HIS/RIS 111. The communication unit 305 transmits the object generated by the output controller 304 to the PACS 112 or the viewer 113.

The display controller 306 controls the display unit 109 so that the display unit 109 displays information. The display controller 306 causes the display unit 109 to display information in accordance with an input from another module or an operation input performed by the user through the operation unit 108. The display controller 306 is an example of display control means.

Series of Processes by Information Processing Apparatus 107

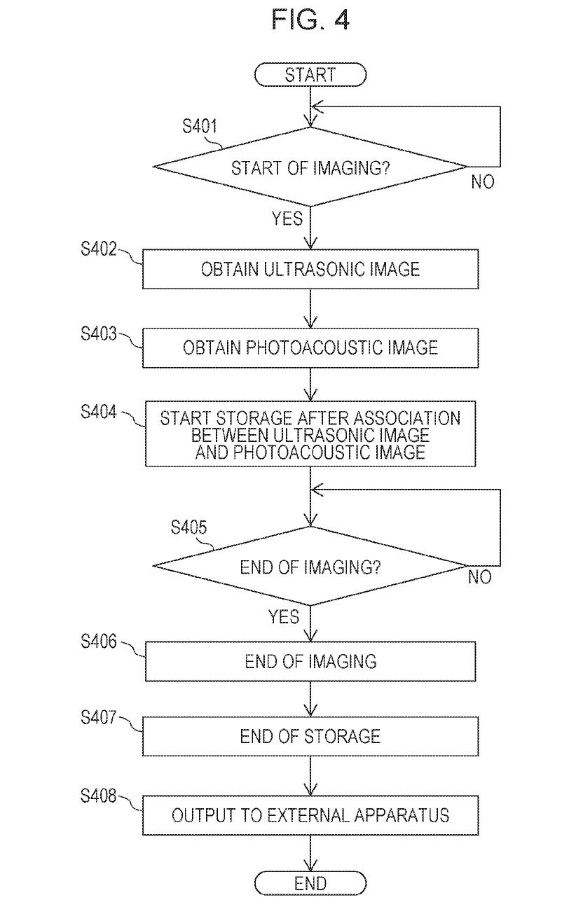

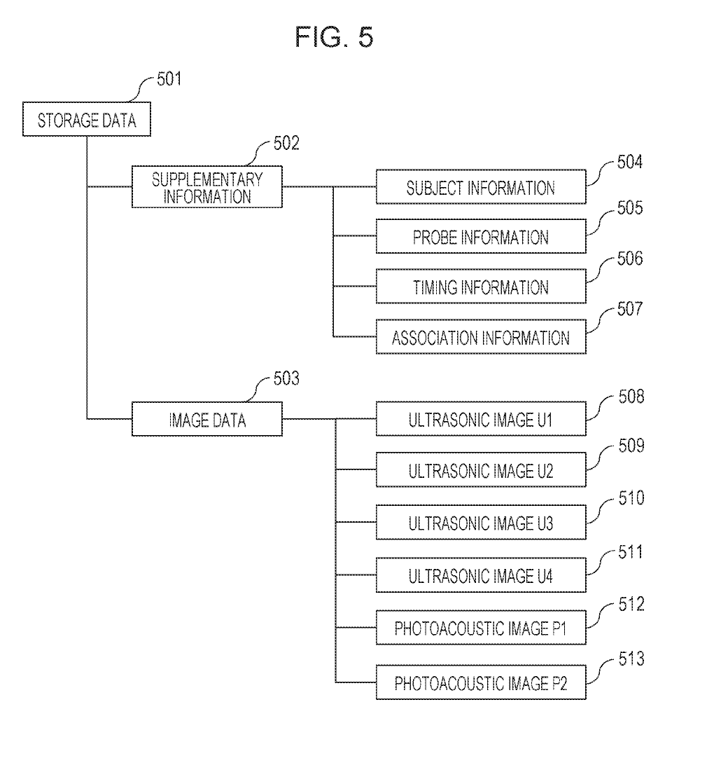

FIG. 4 is a flowchart of an example of a process of capturing a moving image including ultrasonic images and photoacoustic images, generating supplementary information, and outputting an object including the moving image and the supplementary information to an external apparatus. In the process described below, a main component which realizes the processes is the CPU 201 or the GPU unless otherwise noted. Information obtained by the information processing apparatus 107 will also be described appropriately with reference to FIGS. 5 to 7.

In step S401, the inspection controller 301 receives an instruction for starting imaging. First, the inspection controller 301 obtains information on an inspection order from the HIS/RIS 111. The display controller 306 causes the display unit 109 to display information on inspection indicated by the inspection order and a user interface used by the user to input an instruction for performing the inspection. Imaging is started in response to an imaging start instruction input to the user interface through the operation unit 108. Shooting of a moving image including ultrasonic images and photoacoustic images is started based on an operation input performed by the user or is automatically started.

In step S402, the imaging controller 302 controls the probe 103 and the signal collection unit 104 so as to start capturing of an ultrasonic image. The user presses the probe 103 onto the subject 101 so as to perform imaging in a desired position. The imaging controller 302 obtains an ultrasonic signal which is a digital signal and timing information associated with obtainment of the ultrasonic signal to be stored in the RAM 203. The image processor 303 performs a delay-and-sum process and the like on the ultrasonic signal so as to generate an ultrasonic image. Note that, after the ultrasonic image is generated, the ultrasonic signal stored in the RAM 203 may be deleted. The image processor 303 causes the display unit 109 to display the obtained ultrasonic image through the display controller 306. The imaging controller 302 and the image processor 303 repeatedly execute these processes so as to update the ultrasonic image displayed in the display unit 109. In this way, the ultrasonic images are displayed as a moving image.

In step S403, the imaging controller 302 controls the probe 103 and the signal collection unit 104 so as to start capturing of a photoacoustic image. The user presses the probe 103 onto the subject 101 so as to perform imaging in a desired position. The imaging controller 302 obtains a photoacoustic signal which is a digital signal and timing information associated with obtainment of the photoacoustic signal to be stored in the RAM 203. The image processor 303 performs a universal back-projection (UBP) process and the like on the photoacoustic signal so as to generate a photoacoustic image. Note that, after the photoacoustic image is generated, the photoacoustic signal stored in the RAM 203 may be deleted. The image processor 303 causes the display unit 109 to display the obtained photoacoustic image through the display controller 306. The imaging controller 302 and the image processor 303 repeatedly execute these processes so as to update the photoacoustic image displayed in the display unit 109. In this way, the photoacoustic images are displayed as a moving image.

The process in step S402 and the process in step S403 may be simultaneously performed, may be switched from one to another at a predetermined interval, or may be switched based on an operation input performed by the user or an inspection order. Although the case where the capturing of ultrasonic images is performed first is illustrated, the capturing of photoacoustic images may be performed first. The display controller 306 may display the ultrasonic image and the photoacoustic image such that one of the images is superposed on the other or may display the ultrasonic image and the photoacoustic image in parallel in step S402. Furthermore, the image processor 303 may obtain a superposed image by superposing the ultrasonic image and the photoacoustic image on each other, and the display controller 306 may cause the display unit 109 to display the superposed image.

In step S404, the output controller 304 associates the ultrasonic image and the photoacoustic image obtained in step S402 and step S403, respectively, with each other and stores the associated images in the storage device 204 with supplementary information. In step S404, the output controller 304 repeatedly processes each frame of ultrasonic image and each frame of photoacoustic image obtained in step S402 and step S403, respectively, so that a file of a moving image including the ultrasonic images and the photoacoustic images is stored.

FIG. 5 is a diagram illustrating an example of a configuration of data obtained in step S404. Storage data 501 is stored in the storage device 204 in step S404. The storage data 501 includes supplementary information 502 and image data 503. For example, the supplementary information 502 is recorded in a header portion of the storage data 501.

The image data 503 includes ultrasonic images 508 to 511 obtained in step S402 and photoacoustic images 512 and 513 obtained in step S403. In the example of FIG. 5, the ultrasonic images 508 to 511 have identifiers U1 to U4 assigned thereto for uniquely identifying the individual ultrasonic images 508 to 511. Furthermore, the photoacoustic images 512 and 513 have identifiers P1 and P2 assigned thereto for uniquely identifying the individual photoacoustic images 512 and 513.

The supplementary information 502 includes subject information 504 indicating an attribute of the subject 101 and probe information 505 indicating information on the probe 103 used in the imaging.

The subject information 504 includes at least one of a subject ID, a name of the subject, an age, a blood pressure, a heart rate, a body temperature, a body height, a body weight, past illness, the number of weeks of pregnancy, and inspection information, for example. Note that, if the inspection system 102 includes an electrocardiograph (not illustrated) or a pulse oximeter (not illustrated), information associated with an electrocardiogram or a degree of oxygen saturation may be stored as the subject information 504.

The probe information 505 includes information associated with the probe 103, such as a type of the probe 103 and a position and an inclination of the probe 103 at a time of the imaging. The inspection system 102 may include a magnetic sensor (not illustrated) which detects a position and an inclination of the probe 103, and the imaging controller 302 may obtain the information from the magnetic sensor (not illustrated).

The supplementary information 502 includes timing information 506 of ultrasonic signals associated with the ultrasonic images 508 to 511 stored in step S404 and photoacoustic signals associated with the photoacoustic images 512 and 513 stored in step S404.

The timing information 506 is obtained in step S402 and step S403 as described above. The timing information 506 is indicated by a time point or a period of time elapsed after start of the inspection as described above. The timing information of the ultrasonic images associates with timings when the ultrasonic signals used for the ultrasonic images are obtained. The timing information obtained when a plurality of ultrasonic signals are used for a single ultrasonic image corresponds to a timing when an arbitrary one of the ultrasonic signals is obtained as long as the same operation is performed on the ultrasonic images obtained in a single inspection operation. The timing when an ultrasonic signal is obtained may correspond to a timing when the information processing apparatus 107 receives the ultrasonic signal, a timing when the probe 103 transmits an ultrasonic wave to the subject 101, a timing when the probe 103 receives an ultrasonic wave, a timing when a driving signal of transmission/reception of an ultrasonic wave transmitted to the probe 103 is detected, or a timing when the signal collection unit 104 receives an ultrasonic signal. The timing information of the photoacoustic images associates with timings when the photoacoustic signals used for the photoacoustic images are obtained. The timing information obtained when a plurality of photoacoustic signals are used for a single photoacoustic image corresponds to a timing when an arbitrary one of the photoacoustic signals is obtained as long as the same operation is performed on the photoacoustic images obtained in a single inspection operation. The timing when a photoacoustic signal is obtained may correspond to a timing when the information processing apparatus 107 receives the photoacoustic signal, a timing when the probe 103 emits light to the subject 101, a timing when the probe 103 receives a photoacoustic wave, a timing when a driving signal of emission of light or reception of an photoacoustic wave to the probe 103 is detected, or a timing when the signal collection unit 104 receives a photoacoustic signal.

The supplementary information 502 includes association information 507 which associates the ultrasonic images 508 to 511 with the photoacoustic images 512 and 513. The association information 507 includes information on photoacoustic images individually associated with ultrasonic images obtained at a frame rate which is higher than that of the photoacoustic images.

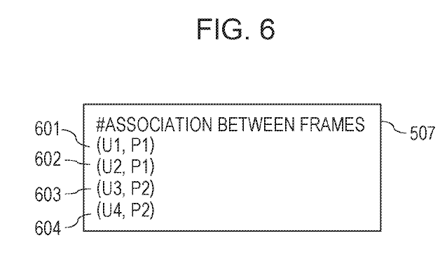

FIG. 6 is a diagram illustrating an example of the association information 507. (Um, Pn) recorded in individual rows 601 to 604 indicates that a frame Pn of a photoacoustic image is associated with a frame Um of an ultrasonic image. In the first embodiment, at least one of frames of the photoacoustic images obtained in a single inspection operation is associated with each of frames of the ultrasonic images obtained in the inspection operation.

In a case where a plurality of photoacoustic images indicating information on an initial sound pressure, information on a light absorption energy density, and information on a light absorption coefficient are obtained from the same photoacoustic signal, frames of the plurality of photoacoustic images may be associated with each of the frames of the ultrasonic images. In this case, frames Px, Py, and Pz of photoacoustic images may be associated with a frame Um of an ultrasonic image and the association is represented as (Um, Px, Py, Pz).

In step S405, the imaging controller 302 receives an instruction for terminating the imaging. In the inspection, the display controller 306 causes the display unit 109 to display a user interface which receives an input of an instruction issued by the user. The imaging controller 302 terminates the imaging in response to the imaging termination instruction input to the user interface through the operation unit 108. Alternatively, the imaging controller 302 may determine that the imaging is to be terminated when a predetermined period of time has elapsed after the imaging start instruction is received in step S401. When the imaging is terminated, the inspection controller 301 transmits information indicating that the imaging is terminated to the HIS/RIS 111 through the communication unit 305.

In step S406, the imaging controller 302 controls the probe 103 so as to terminate the capturing of ultrasonic images and photoacoustic images.

In step S407, the output controller 304 terminates the process of storing ultrasonic images and photoacoustic images started in step S404.

In step S408, the output controller 304 generates an object for outputting the moving image including the ultrasonic images and the photoacoustic images obtained in step S402 and step S403, respectively, based on the information stored until step S407. The communication unit 305 outputs the object to the external apparatus, such as the PACS 112.

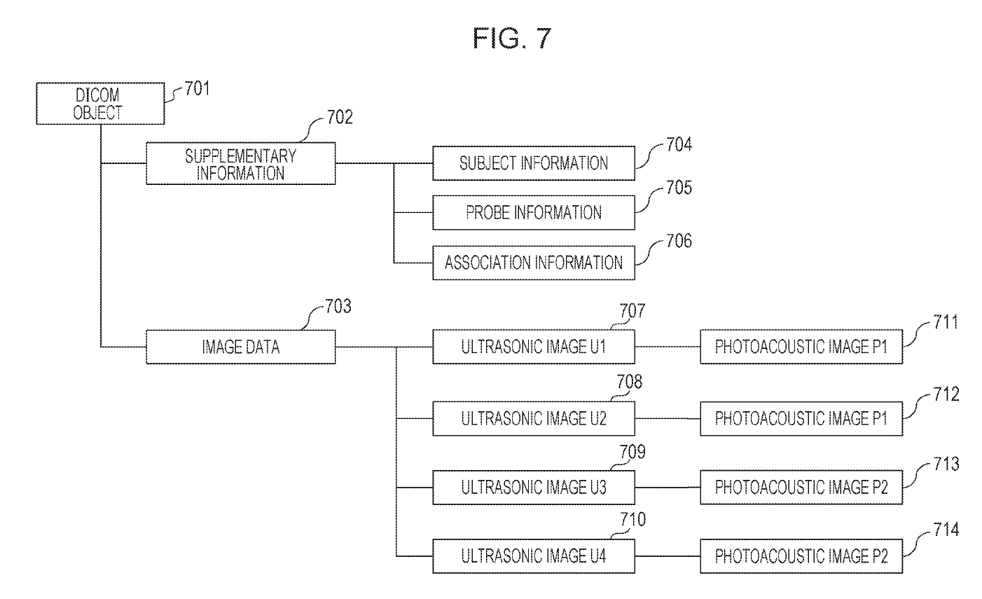

FIG. 7 is a diagram illustrating an example of the object generated in step S408. A DICOM object 701 includes supplementary information 702 and image data 703. In FIG. 7, although the supplementary information 702 and the image data 703 are separately illustrated for simplicity, the supplementary information 702 may be described in a header portion of the image data 703.

The supplementary information 702 includes subject information 704, probe information 705, and association information 706. The subject information 704 corresponds to the subject information 504 of FIG. 5. The probe information 705 corresponds to the probe information 505 of FIG. 5. The association information 706 corresponds to the association information 507 of FIG. 5. The information included in the supplementary information 702 may include information which is the same as that of the corresponding information illustrated in FIG. 5, include only information required for the DICOM standard, or include only predetermined items which are arbitrarily set. For example, the subject information 704 may include only information on a subject ID, information on an age, information on a gender, and information on an inspection ID. The supplementary information 702 may not include the probe information 705. Furthermore, the supplementary information 702 may further include timing information corresponding to the timing information 506 of FIG. 5.

The image data 703 includes ultrasonic images 707 to 710 and photoacoustic images 711 to 714. In the example of FIG. 7, the photoacoustic images 711 to 714 are associated with the ultrasonic images 707 to 710, respectively, as overlay images. Although the photoacoustic images 711 and 712 correspond to the photoacoustic image P1, the photoacoustic image 712 may be changed to the photoacoustic image P1 and only information on the photoacoustic image P1 may be included. Specifically, at least information indicating that a photoacoustic image corresponding to an ultrasonic image 708 corresponds to the photoacoustic image P1 is included in the DICOM object 701. An example of the information is the association information 706.

As another example of the DICOM object 701, the photoacoustic images are separated from the DICOM object 701 so that another DICOM object, such as color softcopy presentation state (CSPS), may be generated. When the CSPS is to be used, the output controller 304 may convert the photoacoustic images into an annotation object.

Furthermore, as another example, a superposed image obtained by superposing an ultrasonic image and a photoacoustic image may be determined as secondary capture.

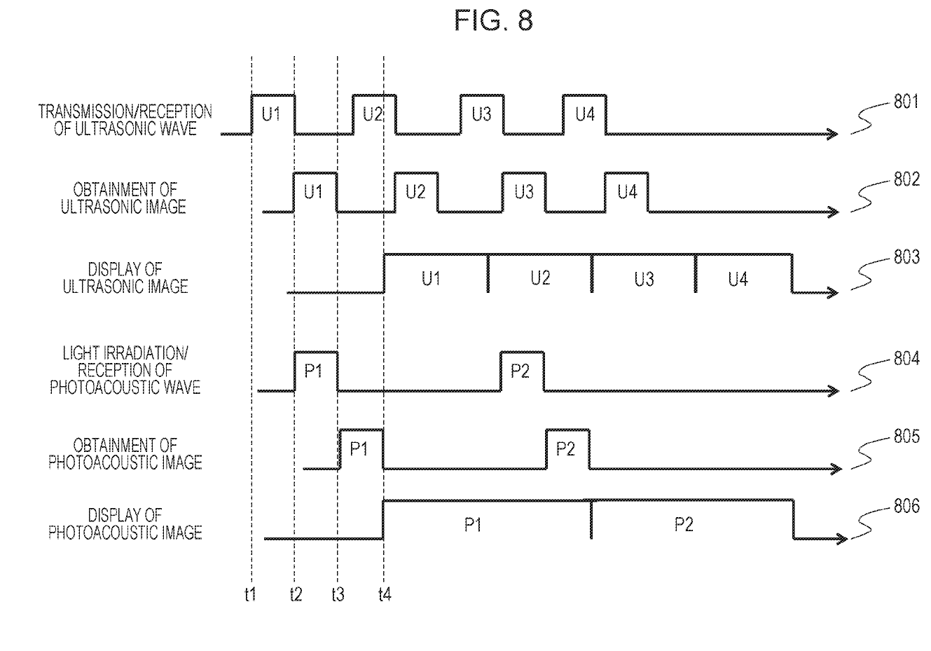

FIG. 8 includes timing charts of a process of obtaining an ultrasonic image and a photoacoustic image. In diagrams 801 to 806, time elapses rightward in the sheet. The individual diagrams rise or fall at time points t1 to t4 in the timing charts. Hereinafter, rises or falls of the diagrams are simply referred to as rises or falls.

The diagram 801 indicates a timing associated with an obtainment of an ultrasonic signal. In a rising portion, the probe 103 starts transmission of an ultrasonic wave to the subject 101 and an obtained reflection wave is appropriately transmitted as an ultrasonic signal to the information processing apparatus 107. In a falling portion, the imaging controller 302 terminates reception of an ultrasonic signal. Frames U1 to U4 correspond to frames of ultrasonic images. It is assumed that, in the frame U1, the probe 103 transmits an ultrasonic wave to the subject 101 at the time point t1, and the imaging controller 302 terminates reception of the ultrasonic wave at the time point t2.

The diagram 802 indicates a timing associated with an obtainment of an ultrasonic signal. In a rising portion, the image processor 303 starts generation of an ultrasonic image. In a falling portion, the image processor 303 completes the generation of an ultrasonic image and the information processing apparatus 107 obtains an ultrasonic image. It is assumed that, in the frame U1, image processor 303 starts generation of an ultrasonic image at the time point t2, and terminates the generation of an ultrasonic image at the time point t3.

The diagram 803 indicates a timing associated with display of an ultrasonic signal. The ultrasonic image may be displayed when the obtainment of the ultrasonic image is completed. The display controller 306 starts display of the frame U1 at the time point t4 and performs display while successively switching the frames in a predetermined rate to the frames U2 to U4. The process in step S402 of FIG. 4 corresponds to the diagrams 801 to 803.

The diagram 804 indicates a timing associated with an obtainment of a photoacoustic signal. In a rising portion, the probe 103 starts emission of light to the subject 101 and an obtained photoacoustic wave is appropriately transmitted as a photoacoustic signal to the information processing apparatus 107. In a falling portion, the imaging controller 302 terminates reception of a photoacoustic signal. Frames P1 and P2 correspond to frames of photoacoustic images. It is assumed that, in the frame P1, the probe 103 emits light to the subject 101 at the time point t2, and the imaging controller 302 terminates reception of the photoacoustic signal at the time point t3.

The diagram 805 indicates a timing associated with an obtainment of a photoacoustic image. In a rising portion, the image processor 303 starts generation of a photoacoustic image. In a falling portion, the image processor 303 terminates the generation of a photoacoustic image and the information processing apparatus 107 obtains a photoacoustic image. It is assumed that, in the frame P1, the image processor 303 starts generation of a photoacoustic image at the time point t3, and terminates the generation of a photoacoustic image at the time point t4.

The diagram 806 indicates a timing associated with display of a photoacoustic image. The photoacoustic image may be displayed when the obtainment of a photoacoustic image is completed. The display controller 306 starts display of the frame P1 at the time point t4 and performs display while switching the frames to the frame P2 in a predetermined rate. The process in step S403 of FIG. 4 corresponds to the diagrams 804 to 806. In the example of FIG. 8, an ultrasonic image is displayed in a frame rate which is twice as high as that of a photoacoustic image.

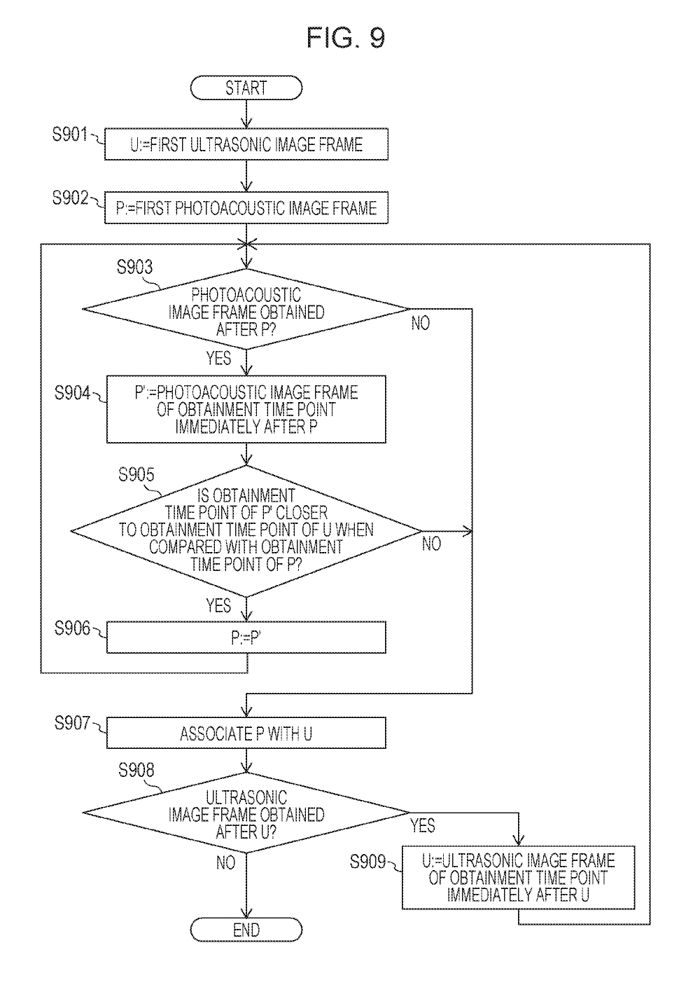

FIG. 9 is a flowchart of an example of a process of associating an ultrasonic image with a photoacoustic image obtained in inspection performed by the output controller 304. Specifically, the process in FIG. 9 is executed in step S404 of FIG. 4. The output controller 304 associates a first photoacoustic image obtained at a first time point or a second photoacoustic image obtained at a second time point after the first time point with an ultrasonic image obtained at a third time point which is included in a period of time from the first time point to the second time point. Specifically, the output controller 304 associates one of the first and second photoacoustic images with the ultrasonic image based on the relationship among the first to third time points. Hereinafter, a case where the photoacoustic image obtained one of the first and the second time points which is closer to the third time point is associated with the ultrasonic image will be described as an example. In the process described below, a main component which realizes operations is CPU 201 or the GPU unless otherwise noted.

In step S901, the output controller 304 sets a temporary variable U indicating a frame of the ultrasonic image to a frame of a first ultrasonic image. The output controller 304 specifies the frame of the first ultrasonic image based on the timing information. The first ultrasonic image is generated based on an ultrasonic signal obtained first, for example. Hereinafter, a time point when an ultrasonic image is obtained is referred to as an obtainment time point of an ultrasonic signal used for generating the ultrasonic image.

In step S902, the output controller 304 sets a temporary variable P indicating a frame of a photoacoustic image to a frame of a first photoacoustic image. The output controller 304 specifies the frame of the first photoacoustic image based on the timing information. The first photoacoustic image is generated based on a photoacoustic signal obtained first, for example.

Hereinafter, a time point when a photoacoustic image is obtained is referred to as an obtainment time point of a photoacoustic signal used for generating the photoacoustic image.

In step S903 to step S909 described below, the output controller 304 obtains, for each of frames U of ultrasonic images, a frame P of a photoacoustic image having an obtainment time point which is closest to an obtainment time point of the frame U and associates the frame P with the frame U. Specifically, in the first embodiment, an ultrasonic image is associated with a photoacoustic image of an obtainment time point closest to that of the ultrasonic image.

In step S903, the output controller 304 obtains information on a frame of a photoacoustic image obtained at a time point after the obtainment time point of the frame P set in step S902. When a frame is obtained after the obtainment time point of the frame P, the process proceeds to step S904, and otherwise, the process proceeds to step S907. The obtainment time point of the frame P is an example of the first time point.

In step S904, the output controller 304 sets a temporary variable P′ indicating a frame of a photoacoustic image as a frame of a photoacoustic image obtained immediately after the frame P. The obtainment time point of the frame P′ is an example of the second time point.

In step S905, the output controller 304 compares the obtainment time point of the frame P′ with the obtainment time point of the frame P. In a case where an obtainment time point tp′ of the frame P′ is closer to an obtainment time point to of the frame U when compared with an obtainment time point tp of the frame P, that is, an expression “|tp′−tu|<|tp−tu| is satisfied, the process proceeds to step S906, and otherwise, the process proceeds to step S907.

In step S906, the output controller 304 sets the temporary variable P indicating a frame of a photoacoustic image to the frame indicated by P′ set in step S904, and the process proceeds to step S903.

In step S907, the output controller 304 associates the frame P of the photoacoustic image to the frame U of the ultrasonic image, and the process proceeds to step S908.

In step S908, the output controller 304 obtains information on a frame of an ultrasonic image obtained at the time point after the obtainment time point of the frame U. When a frame is obtained after the obtainment time point of the frame U, the process proceeds to step S909, and otherwise, the process in FIG. 9 is terminated.

In step S909, the output controller 304 sets the temporary variable U indicating a frame of an ultrasonic image to a frame of an ultrasonic image obtained immediately after the ultrasonic image corresponding to the frame U set in step S901 or step S909 in an immediately preceding loop. Thereafter, the process proceeds to step S903.

According to the process described above, if a photoacoustic image is not obtained before the frame U, a photoacoustic image having a first obtainment time point is associated with the ultrasonic image. In this case, the obtainment time point of the ultrasonic image and the obtainment time point of the first photoacoustic image may be far from each other. Association between images having distant obtainment time points may be inappropriate when a doctor observes a moving image. When the determination in step S905 is negative, the output controller 304 may obtain a value of tp−tu. When the resultant value is larger than a predetermined threshold value, the frame P and the frame U may not be associated with each other in step S907.

According to the process described above, if a photoacoustic image is not obtained after the frame U, a photoacoustic image having a last obtainment time point is associated with the ultrasonic image. In this case, the obtainment time point of the ultrasonic image and the last obtainment time point of the photoacoustic image may be far from each other. Association between images having distant obtainment time points may be inappropriate when a doctor observes a moving image. When the determination in step S903 is negative, the output controller 304 may obtain a value of tu-tp. When the resultant value is larger than a predetermined threshold value, the frame P and the frame U may not be associated with each other in step S907.

According to the process described above, when photoacoustic images are obtained before and after the obtainment time point of the frame U, one of the photoacoustic images obtained before and after the obtainment time point of the frame U which is closer to the obtainment time point of the frame U is associated with the ultrasonic image. The obtainment time point of the frame U is an example of the third time point included in the period between the first and second time points.

Note that, although an example of the process performed when a frame of a photoacoustic image is associated with a frame of an ultrasonic image one by one is illustrated in FIG. 9, the output controller 304 may perform a process of associating frames of a plurality of types of photoacoustic image with each of frames of ultrasonic images. The viewer 113 may display a moving image associated with frames of a plurality of types of photoacoustic image such that the different types of photoacoustic image are superposed on one another.

Furthermore, the process performed when the frame P of the photoacoustic image having the obtainment time point which is closest to the obtainment time point of the frame U of the ultrasonic image is associated is illustrated as an example in FIG. 9. However, the output controller 304 may associate a frame of a photoacoustic image having an obtainment time point immediately before or immediately after the obtainment time point of the frame U with the ultrasonic image. Specifically, a frame of a photoacoustic image having an obtainment time point within a predetermined period of time before and after the obtainment time point of the frame U may be associated irrespective of a result of a determination as to whether the obtainment time point of the ultrasonic image is closest to the obtainment time point of the frame U of the ultrasonic image. Alternatively, the image processor 303 generates an interpolation frame P using frames of a plurality of photoacoustic images obtained before and after the obtainment time point of the ultrasonic image frame U and the output controller 304 may associate the interpolation frame P with the frame U. The image processor 303 may generate the interpolation frame P only when a difference between the two photoacoustic images is equal to or smaller than a threshold value.

When the output controller 304 processes the ultrasonic images and the photoacoustic images obtained at the timings illustrated in FIG. 8 in accordance with the flowchart of FIG. 9, the photoacoustic image P1 is associated with the ultrasonic images U1 and U2 and the photoacoustic image P2 is associated with the ultrasonic images U3 and U4. In step S402 and step S403 in FIG. 4, the display controller 306 causes the display unit 109 to display a superposed image obtained by superposing the photoacoustic image P1 on the ultrasonic image U1 or the ultrasonic image U2. In step S404, the output controller 304 associates the photoacoustic image frame P1 with the ultrasonic image frames U1 and U2 to be stored in the association information 507. In step S408, the communication unit 305 transmits the DICOM object 701 having the image data 703 including the association to the PACS 112.

With the configuration of the first embodiment, the frames of the photoacoustic images are associated with the frames of the ultrasonic images. Furthermore, the images may be associated with each other by reducing the frame rate of the ultrasonic images so that the frame rate of the ultrasonic images coincide with the frame rate of the photoacoustic image. However, if the frame rate of the ultrasonic image is reduced, quality of the moving image is degraded. However, according to this embodiment, the images may be associated with each other while the frame rate of the ultrasonic images is maintained. The user displays the DICOM object 701 using the viewer 113 connected to the PACS 112. In this case, the viewer 113 may simultaneously display the ultrasonic image and the photoacoustic image based on the association information 706 included in the DICOM object 701. Since at least one photoacoustic image is associated with each one of the photoacoustic images, flicker of display of the moving image may be reduced even in a case where frame rates of the ultrasonic images and the photoacoustic image are different from each other.

### Second Embodiment

In a second embodiment, a process of receiving an instruction for storing an ultrasonic image and a photoacoustic image is performed, and thereafter, a process of associating the ultrasonic image and the photoacoustic image with each other based on timing information and the instruction is performed.

A configuration of an inspection system 102 including an information processing apparatus 107, a hardware configuration of the information processing apparatus 107, and a functional configuration of the information processing apparatus 107 according to the second embodiment are the same as those illustrated in FIGS. 1 to 3. Detailed descriptions in the common portions will be omitted by employing the descriptions above.

According to the second embodiment, an output controller 304 incorporates a timing of a storage instruction input by a user through an operation unit 108 in the timing information to be stored and further stores image data and supplementary information in response to the instruction. Furthermore, an image processor 303 may transmit the storage instruction to the output controller 304 in response to an obtainment of image data, and the output controller 304 may store the supplementary information including the timing information described above and the image data in response to the instruction issued by the image processor 303.

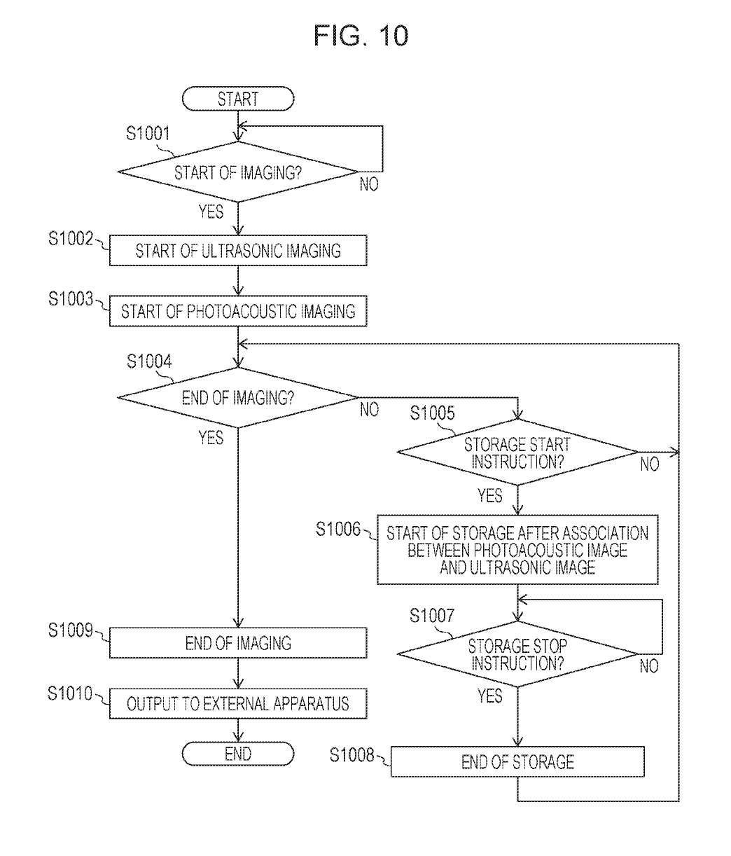

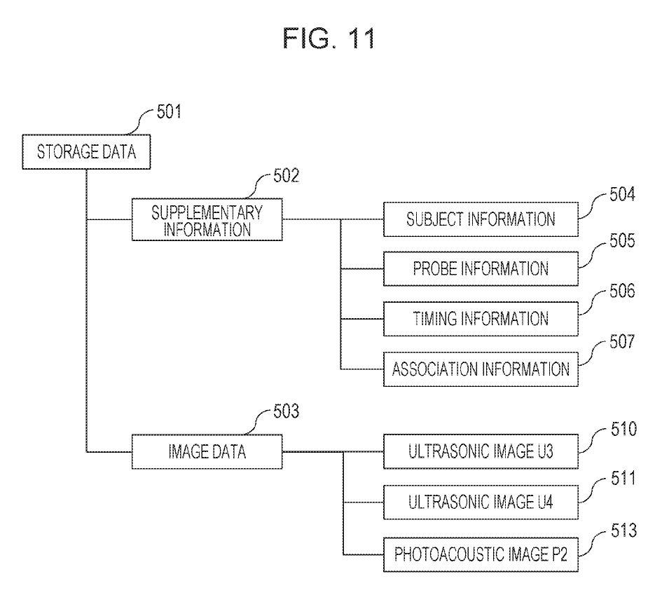

FIG. 10 is a flowchart of an example of a process of capturing a moving image including ultrasonic images and photoacoustic images, generating supplementary information based on a storage instruction, and outputting an object including the moving image and the supplementary information to an external apparatus. In the process described below, a main component which realizes operations are a CPU 201 or a GPU unless otherwise noted. Information obtained by the information processing apparatus 107 will now be described with reference to FIGS. 11 to 13 where appropriate.

A process from step S1001 to step S1003 is the same as that from step S401 to step S403 illustrated in FIG. 4.

In step S1004, the imaging controller 302 receives an instruction for terminating imaging. The imaging controller 302 receives the instruction in a process the same as the process in step S405 of FIG. 4. When the imaging controller 302 receives the instruction, the process proceeds to step S1009 where an inspection controller 301 transmits information indicating that the imaging has been terminated to an HIS/RIS 111 through a communication unit 305. When the instruction for terminating imaging has not been issued, the process proceeds to step S1005.

In step S1005, the output controller 304 receives an instruction for starting storage. The user may input the instruction through a user interface displayed by a display controller 306 in a display unit 109 during inspection. The user may input the instruction for starting storage to the output controller 304 through an input unit (not illustrated), such as a freeze button, disposed on a probe 103. When the output controller 304 receives the instruction, the process proceeds to step S1006, and otherwise, the process proceeds to step S1004.

In step S1006, the output controller 304 associates ultrasonic images and photoacoustic images obtained in step S1002 and step S1003, respectively, with each other so as to store the ultrasonic images and the photoacoustic images in a storage device 204 with supplementary information. In step S1006, the output controller 304 processes the ultrasonic images of frames and the photoacoustic images of frames obtained in step S1002 and step S1003, respectively, so as to store the images as a file of a moving image including the ultrasonic images and the photoacoustic images. Also after step S1004 onwards, the process in step S1002 and step S1003 may be continuously performed so that the process described above is performed on ultrasonic images and photoacoustic images newly obtained in step S1006.

FIG. 11 is a diagram illustrating an example of a configuration of data obtained in step S1006. Components the same as those in FIG. 5 are denoted by reference numerals which are the same as those in FIG. 5, and therefore, the descriptions described above are employed and detailed descriptions thereof are omitted. Timing information 506 further includes information on a timing when a storage instruction is issued.

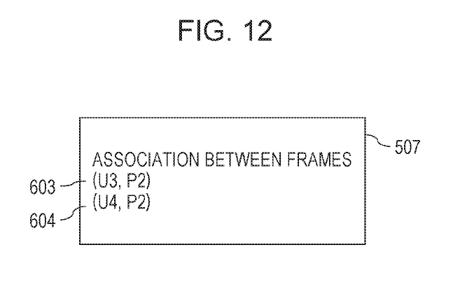

FIG. 12 is a diagram illustrating an example of association information 507 of FIG. 11. Components the same as those in FIG. 6 are denoted by reference numerals which are the same as those in FIG. 6, and therefore, the descriptions described above are employed and detailed descriptions thereof are omitted. In the example of FIG. 12, information on association between frames after the storage instruction is described.

In step S1007, the output controller 304 receives an instruction for stopping the storage. When the output controller 304 receives the storage stop instruction, the process proceeds to step S1008. The user may input the instruction through a user interface displayed by the display controller 306 in the display unit 109 during the inspection. The user may input the instruction through the input unit (not illustrated), such as a freeze button, disposed on the probe 103. When the output controller 304 receives the instruction, the process proceeds to step S1008.

In step S1008, the output controller 304 terminates the process of storing ultrasonic images and photoacoustic images started in step S1006.

In step S1009, the imaging controller 302 controls the probe 103 so as to terminate capturing of an ultrasonic image and a photoacoustic image.

In step S1010, the output controller 304 generates an object for outputting a moving image including the ultrasonic images and the photoacoustic images obtained in step S1002 and step S1003, respectively, to the external apparatus based on the information stored until step S1009. The communication unit 305 outputs the object to the external apparatus, such as a PACS 112.

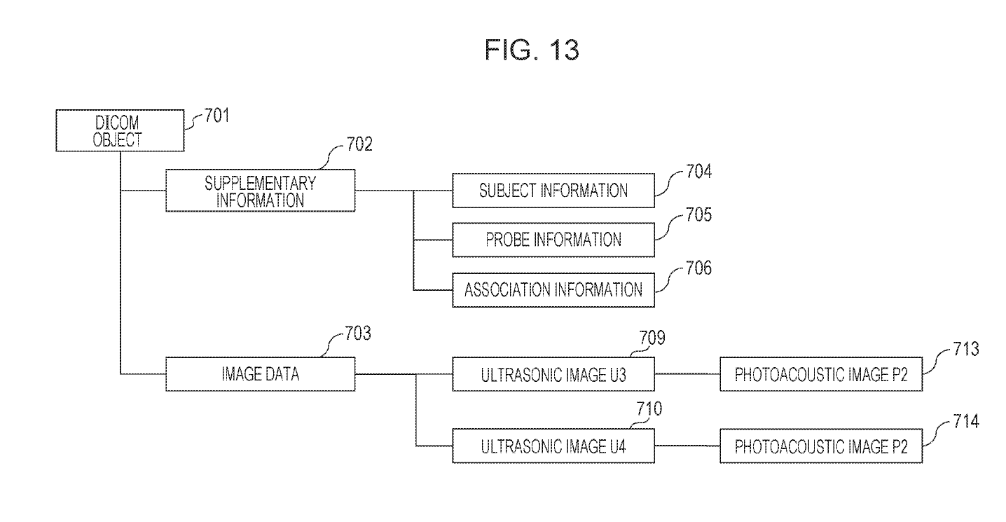

FIG. 13 is a diagram illustrating an example of the object generated in step S1010. Components the same as those in FIG. 7 are denoted by reference numerals which are the same as those in FIG. 7, and therefore, the descriptions described above are employed and detailed descriptions thereof are omitted. The supplementary information 702 may further include information on the timing when the storage instruction is issued.

Note that a series of a process of starting storage and a process of stopping the storage may be repeatedly instructed after imaging is started. In this case, the process in step S1004 to step S1008 is executed every time the storage instruction is input. The output controller 304 may store different storage data 501 in the storage device 204 every time the process in step S1006 is executed or may store single storage data 501 through the repetition. In the case where different storage data 501 is stored every time the process in step S1006 is executed, the output controller 304 may convert single storage data 501 into a single DICOM object 701 in step S1010. Alternatively, the output controller 304 may convert a plurality of storage data 501 into a single DICOM object 701.

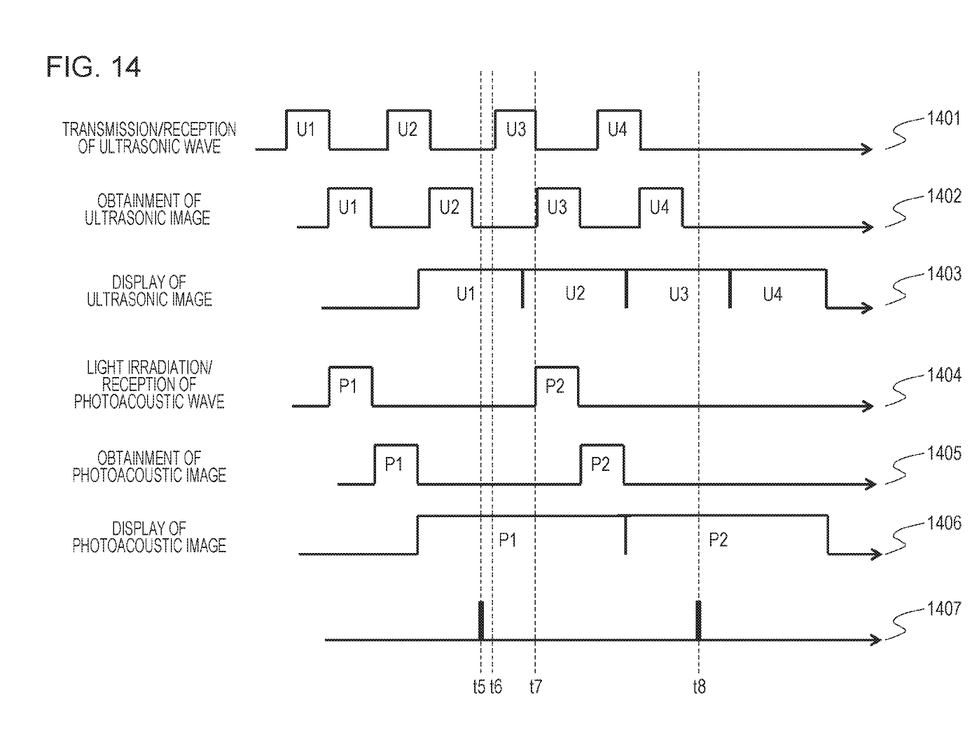

FIG. 14 includes timing charts of a process of obtaining an ultrasonic image and a photoacoustic image. In diagrams 1401 to 1407, time elapses rightward in the sheet. Time points t5 to t8 indicate time points of rises and falls in the timing charts.

The individual diagrams 1401 to 1406 associate with the processes of the diagrams 801 to 806, respectively, in FIG. 8. The probe 103 transmits an ultrasonic wave to a subject 101 at the time point t6, and an imaging controller 302 terminates reception of an ultrasonic signal at the time point t7. The probe 103 starts emission of light to the subject 101 at the time point t7.

The diagram 1407 indicates a timing when a storage instruction is issued. The imaging controller 302 receives an instruction for starting storage based on an operation input performed by a user at the time point t5, for example, and receives an instruction for terminating the storage at the time point t8.

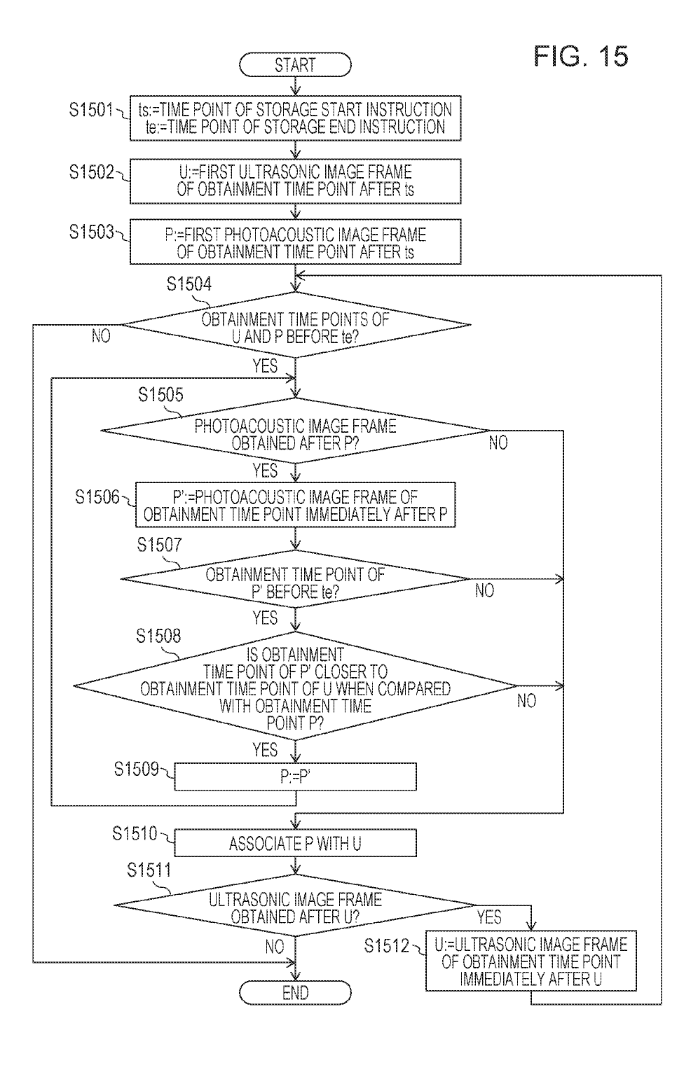

FIG. 15 is a flowchart of an example of a process of associating an ultrasonic image with a photoacoustic image obtained in the inspection performed by the output controller 304. Specifically, the process in FIG. 15 is executed in step S1006 of FIG. 10. In the process described below, a main component which realizes operations is the CPU 201 or the GPU unless otherwise noted.

In step S1501, the output controller 304 sets variables ts and te indicating time points to a time point when an instruction for starting storage is received and a time point when an instruction for terminating the storage is received, respectively. Note that when the output controller 304 has not received an instruction for terminating the storage, the variable te may not be set or an arbitrary value which attains an affirmative determination in step S1504 described below may be set.

In step S1502, a temporary variable U indicating a frame of an ultrasonic image is set to a frame of a first ultrasonic image after an obtainment time point ts. The output controller 304 specifies the frame of the first ultrasonic image after the obtainment time point ts based on timing information.

In step S1503, a temporary variable P indicating a frame of a photoacoustic image is set as a frame of a first photoacoustic image after the obtainment time point ts. The output controller 304 specifies the frame of the first photoacoustic image after the obtainment time point ts based on the timing information.

In step S1504 to step S1512 described below, the output controller 304 obtains, for each of frames U of ultrasonic images captured in a period of time from the time point ts to the time point te, a frame P of a photoacoustic image which is captured in the period of time from the time point ts to the time point te and which has an obtainment time point closest to an obtainment time point of the frame U. Then the frame P is associated with the frame U.

In step S1504, the output controller 304 obtains information on the obtainment time points of the frames U and P. When the obtainment time points are before the time point te, the process proceeds to step S1505 and when at least one of the obtainment time points is after the obtainment time point te, the process in FIG. 15 is terminated. Note that, when the output controller 304 has not received the instruction for terminating the storage, and therefore, a value of the variable te has not been set, the process proceeds to step S1505.

In step S1505, the output controller 304 obtains information on a frame of a photoacoustic image obtained at a time point after the obtainment time point of the frame P set in step S1503. When a frame is obtained after the obtainment time point of the frame P, the process proceeds to step S1506, and otherwise, the process proceeds to step S1510.

In step S1506, the output controller 304 sets a temporary variable P′ indicating a frame of a photoacoustic image as a frame of a photoacoustic image obtained immediately after the frame P.

In step S1507, the output controller 304 obtains information on an obtainment time point of a frame P′. When the obtainment time point of the frame P′ is before the time point te, the process proceeds to step S1508, and otherwise, the process proceeds to step S1510. Note that, when the output controller 304 has not received the instruction for terminating the storage, and therefore, a value of the variable te has not been set, the process proceeds to step S1508.

In step S1508, the output controller 304 compares the obtainment time point of the frame P′ with the obtainment time point of the frame P. When a obtainment time point tp′ of the frame P′ is closer to an obtainment time point to of the frame U when compared with an obtainment time point tp of the frame P, that is, an expression “|tp′−tu|<|tp−tu| is satisfied, the process proceeds to step S1509, and otherwise, the process proceeds to step S1510.

In step S1509, the output controller 304 sets the temporary variable P indicating a frame of a photoacoustic image to the frame P′ set in step S1506, and the process proceeds to step S1505.

In step S1510, the output controller 304 associates the frame P of the photoacoustic image with the frame U of the ultrasonic image, and the process proceeds to step S1511.

In step S1511, the output controller 304 obtains information on the frame of the ultrasonic image obtained at the time point after the obtainment time point of the frame U. When a frame is obtained after the obtainment time point of the frame U, the process proceeds to step S1512, and otherwise, the process in FIG. 15 is terminated.

In step S1512, the output controller 304 sets the temporary variable U indicating a frame of an ultrasonic image to a frame of an ultrasonic image obtained immediately after the ultrasonic image set to the frame U in step S1502 or in step S1512 in an immediately preceding loop. Thereafter, the process proceeds to step S1504.

When the output controller 304 processes the ultrasonic images and the photoacoustic images obtained at the timings illustrated in FIG. 14 in accordance with the flow of FIG. 15, a photoacoustic image P2 is associated with ultrasonic images U3 and U4 which are obtained after the storage start instruction. In step S1003 of FIG. 10, the display controller 306 causes the display unit 109 to display a superposed image obtained by superposing the photoacoustic image P1 on the ultrasonic image U1 or the ultrasonic image U2. Furthermore, the display controller 306 causes the display unit 109 to display a superposed image obtained by superposing the photoacoustic image P2 on the ultrasonic image U3 or the ultrasonic image U4. In step S1006, the output controller 304 associates the ultrasonic images U3 and U4 which are captured in the period of time from the time point is when the storage start instruction is issued to the time point to when the storage termination instruction is issued with the photoacoustic image frame P2 and stores the association in the association information 507. In step S1011, the communication unit 305 transmits the DICOM object 701 having the image data 703 including the association to the PACS 112.

According to the configuration of the second embodiment, the user may capture ultrasonic images and a photoacoustic image and associate a frame of the photoacoustic image with frames of the ultrasonic images obtained in a period of time in which storage is instructed while observing a superposed image displayed in the display unit 109. When the DICOM object 701 is reproduced by the viewer 113, flicker in display of a moving image may be reduced even in a case where frame rates of the ultrasonic images and the photoacoustic images are different from each other.

### Third Embodiment

According to a third embodiment, in a case where a photoacoustic image is included in a section of a moving image constituted by a series of ultrasonic images, the section including the photoacoustic image is quickly specified at a time of reproduction of the moving image based on information on an operation input performed by a user.

A configuration of an inspection system 102 including an information processing apparatus 107, a hardware configuration of the information processing apparatus 107, and a functional configuration of the information processing apparatus 107 according to the third embodiment are the same as those illustrated in FIGS. 1 to 3. Detailed descriptions in the common portions will be omitted by employing the descriptions above.

In the third embodiment, an imaging controller 302 obtains information on an operation input performed by the user (hereinafter referred to as “operation information”) based on information supplied from a probe 103 and information input through an operation unit 108.

Furthermore, according to the third embodiment, an output controller 304 obtains supplementary information including the operation information.

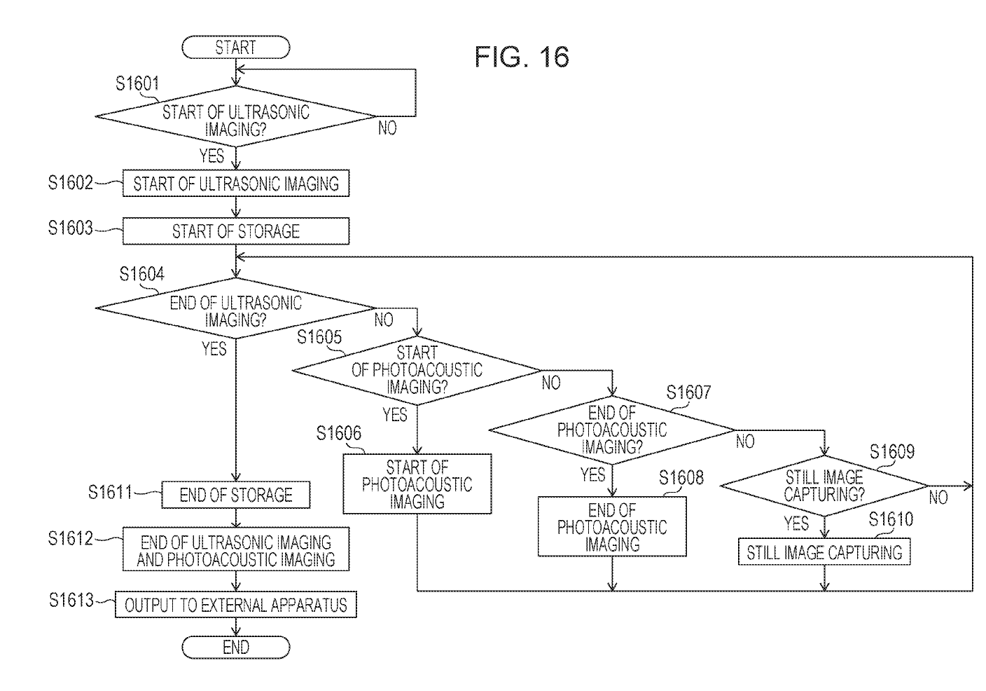

FIG. 16 is a flowchart of an example of a process of capturing a moving image including ultrasonic images and photoacoustic images, generating supplementary information, and outputting an object including the moving image and the supplementary information to an external apparatus. Hereinafter, a case where, when an ultrasonic image is captured, a photoacoustic image is also captured based on an operation input performed by the user will be described as an example. In the process described below, a main component which realizes the processes is a CPU 201 or a GPU unless otherwise noted. Information obtained by the information processing apparatus 107 will now be described with reference to FIGS. 5, 6, 17, 18, and 9 where appropriate.

In step S1601, an inspection controller 301 receives an instruction for starting imaging. First, the inspection controller 301 obtains information on an inspection order from an HIS/RIS 111. A display controller 306 causes a display unit 109 to display information on inspection indicated by the inspection order and a user interface used by the user to input an instruction for performing the inspection. Imaging is started in response to an imaging start instruction input to the user interface through the operation unit 108. Imaging of ultrasonic images is started based on an operation input performed by the user or automatically started.

In step S1602, the imaging controller 302 controls the probe 103 and a signal collection unit 104 so as to start capturing of ultrasonic images. The user presses the probe 103 onto the subject 101 so as to perform imaging in a desired position. The imaging controller 302 obtains ultrasonic signals which are digital signals and timing information associated with an obtainment of the ultrasonic signals to be stored in a RAM 203. An image processor 303 performs a delay-and-sum process and the like on the ultrasonic signals so as to generate ultrasonic images. Note that the ultrasonic signals stored in the RAM 203 may be deleted when the ultrasonic images are generated. The image processor 303 causes the display unit 109 to display the obtained ultrasonic image through the display controller 306. The imaging controller 302 and the image processor 303 repeatedly execute these processes so as to update the ultrasonic image displayed in the display unit 109. In this way, the ultrasonic images are displayed as the moving image.

In step S1603, the output controller 304 starts a process of storing image data obtained by the image processor 303 and supplementary information. An instruction for starting storage is issued by an operation input performed on the information processing apparatus 107 or the probe 103 as described in the second embodiment, for example.

In step S1604, the imaging controller 302 receives an instruction for terminating the ultrasonic imaging. During inspection, the display controller 306 causes the display unit 109 to display a user interface for performing operation inputs associated with the inspection. The user may instruct termination of the ultrasonic imaging by performing an operation input on the user interface. As another example, the user may instruct termination of the ultrasonic imaging by performing an operation input on an input unit (not illustrated) of the probe 103. When the termination instruction is received, the process proceeds to step S1611, and otherwise, the process proceeds to step S1605.

In step S1605, the imaging controller 302 receives an instruction for starting photoacoustic imaging. The user may issue an instruction for starting photoacoustic imaging by performing an operation input to the user interface associated with the inspection or the probe 103. When the start instruction is received, the process proceeds to step S1606, and otherwise, the process proceeds to step S1607.

In step S1604 and step S1605, the imaging controller 302 receives an operation input performed by the user for instructing storage of an ultrasonic image and a photoacoustic image. In this viewpoint, the imaging controller 302 is an example of reception means.

In step S1606, the imaging controller 302 controls the probe 103 and the signal collection unit 104 so as to start capturing of photoacoustic images. The user presses the probe 103 onto the subject 101 so as to perform imaging in a desired position. The imaging controller 302 obtains a photoacoustic signal which is a digital signal and timing information associated with an obtainment of the photoacoustic signal to be stored in the RAM 203. The image processor 303 performs a universal back-projection (UBP) process and the like on the photoacoustic signal so as to generate a photoacoustic image. Note that the ultrasonic signal stored in the RAM 203 may be deleted when the photoacoustic image is generated. The image processor 303 causes the display unit 109 to display the obtained photoacoustic image through the display controller 306. The imaging controller 302 and the image processor 303 repeatedly execute these processes so as to update the photoacoustic image displayed in the display unit 109. In this way, the photoacoustic images are displayed as the moving image. Note that, when the process proceeds from step S1606 to step S1604 where the imaging controller 302 receives an instruction for terminating the ultrasonic imaging, the imaging controller 302 controls the probe 103 so that the capturing of a photoacoustic image is terminated.

In step S1607, the imaging controller 302 receives an instruction for terminating photoacoustic imaging. The user may issue an instruction for terminating photoacoustic imaging by performing an operation input to the user interface associated with the inspection or the probe 103. When the termination instruction is received, the process proceeds to step S1608, and otherwise, the process proceeds to step S1609.

Since the user performs an operation input associated with the capturing of a photoacoustic image in step S1605 and step S1607, the imaging controller 302 obtains operation information.

In step S1608, the imaging controller 302 controls the probe 103 so as to terminate the capturing of a photoacoustic image.

In step S1609, the imaging controller 302 receives an instruction for capturing a still image. The user may issue an instruction for performing still-image capturing by performing an operation input to the user interface associated with the inspection or the probe 103. Here, the still image may be an ultrasonic image, a photoacoustic image, or a superposed image obtained by superposing a photoacoustic image on an ultrasonic image. When the instruction for capturing a still image is received, the process proceeds to step S1610, and otherwise, the process proceeds to step S1604.

In step S1610, the imaging controller 302 controls the probe 103 and the signal collection unit 104 so as to execute a process of capturing still images. The imaging controller 302 controls the probe 103 and the signal collection unit 104 under conditions of a unique operation mode and a unique sampling cycle for capturing still images. The process of obtaining an ultrasonic image and a photoacoustic image performed by the image processor 303 is the same as that described in step S1602 and step S1608.

In the process from step S1604 to step S1610, the imaging controller 302 obtains timing information of the ultrasonic images and the photoacoustic images. The timing information of the ultrasonic images associates with timings when the ultrasonic signals used for the ultrasonic images are obtained. The timing information obtained when a plurality of ultrasonic signals are used for a single ultrasonic image is associated with a timing when the arbitrary ultrasonic signal is obtained, and the same operation is performed for the ultrasonic images obtained in a single inspection operation. The timing when an ultrasonic signal is obtained may correspond to a timing when the information processing apparatus 107 receives the ultrasonic signal, a timing when the probe 103 transmits an ultrasonic wave to the subject 101, a timing when the probe 103 receives an ultrasonic wave, a timing when a driving signal of transmission/reception of an ultrasonic wave relative to the probe 103 is detected, or a timing when the signal collection unit 104 receives an ultrasonic signal. The timing information of the photoacoustic images associates with timings when the photoacoustic signals used for the photoacoustic images are obtained. The timing information obtained when a plurality of photoacoustic signals are used for a single photoacoustic image is associated with a timing when an arbitrary photoacoustic signal is obtained, and the same operation is performed for the photoacoustic images obtained in a single inspection operation. The timing when a photoacoustic signal is obtained may correspond to a timing when the information processing apparatus 107 receives the photoacoustic signal, a timing when the probe 103 emits light to the subject 101, a timing when the probe 103 receives a photoacoustic wave, a timing when a driving signal for the probe 103 which performs emission of light or reception of an photoacoustic wave is detected, or a timing when the signal collection unit 104 receives a photoacoustic signal.

The output controller 304 performs a process of associating photoacoustic images with ultrasonic images when the ultrasonic images and the photoacoustic image are captured by the process described in the first and second embodiments.

In step S1611, the output controller 304 stores information obtained in a period of time from step S1603 to step S1611 and terminates a process of the storage.

FIG. 5 is a diagram illustrating an example of a configuration of data obtained in the process of the storage which is started in step S1603 and terminated in step S1611. Storage data 501 is stored in the storage device 204. The storage data 501 includes supplementary information 502 and image data 503. For example, the supplementary information 502 is recorded in a header portion of the storage data 501.

The supplementary information 502 includes subject information 504 indicating an attribute of a subject 101, probe information 505 indicating information on the probe 103 used in imaging, timing information 506, and association information 507. FIG. 6 is a diagram illustrating an example of the timing information 506.

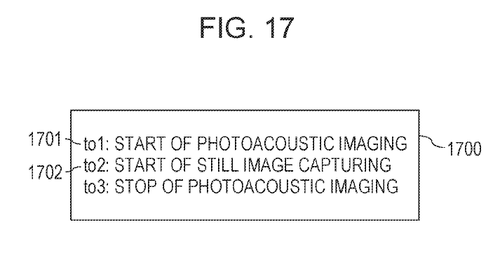

In the third embodiment, the supplementary information 502 further includes operation information 1700 (FIG. 17).

FIG. 17 is a diagram illustrating an example of the operation information 1700. Individual rows in the operation information 1700 include a time point and content of an operation instructed at the time point which are recorded in time series. For example, a line 1701 indicates that an instruction for starting photoacoustic imaging has been issued at a time point tot. Furthermore, a line 1702 indicates that an instruction for starting still-image capturing has been issued at a time point to2. A timing when the user has performed an operation input using the operation unit 108, for example, is recorded as an instruction time point.

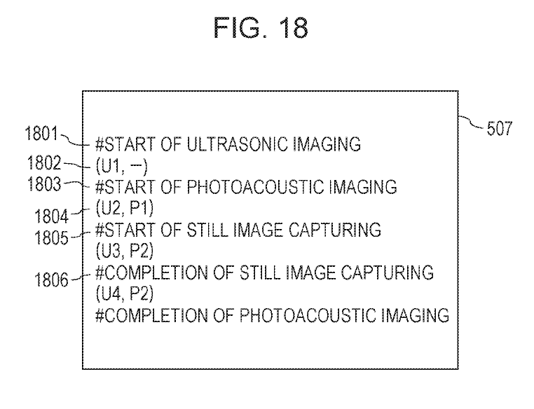

FIG. 18 is a diagram illustrating an example of the association information 507. In individual rows in the association information 507, an operation input performed by the user or an identifier of an obtained image are recorded in time series. (Um, Pn) indicates that a frame Um of an ultrasonic image and a frame Pn of a photoacoustic image are associated with each other by the process illustrated in the first embodiment or the process illustrated in the second embodiment. (Um, −) indicates that only a frame Um of an ultrasonic image is obtained at a certain timing. A row starting with a mark “#” indicates content of an operation input performed by the user. For example, in rows 1801 to 1804, a frame U1 of an ultrasonic image is obtained after an instruction for starting ultrasonic imaging, and thereafter, an instruction for starting photoacoustic imaging has been issued.

The association information 507 may include virtual operation inputs which are not performed by operation inputs by the user. The virtual operation input is automatically performed by the apparatus, and indicates a logical event, such as an interim progress of the process or completion of the process, in a case where the information processing apparatus 107 executes a series of processes using an operation input performed by the user as a trigger. For example, “#completion of still image capturing” in the line 1806 is a virtual operation input and indicates completion of a process of capturing a still image which is executed using an instruction for starting still-image capturing in the line 1805 as a trigger. The virtual operation input is automatically inserted by the output controller 304 into the association information 507.

In step S1612, the imaging controller 302 controls the probe 103 so as to terminate capturing of ultrasonic images and photoacoustic images.

In step S1613, the output controller 304 generates an object to be output to an external apparatus based on information stored until step S1611. The communication unit 305 outputs the object to the external apparatus, such as the PACS 112.

FIG. 7 is a diagram illustrating an example of the object generated in step S413. A DICOM object 701 includes supplementary information 702 and image data 703. For example, the supplementary information 702 is recorded in a header portion of image data 703.

The supplementary information 702 includes subject information 704, probe information 705, and association information 706. The subject information 704 corresponds to the subject information 504 of FIG. 5. The probe information 705 corresponds to the probe information 505 of FIG. 5. The association information 706 corresponds to the association information 507 of FIG. 18. The information included in the supplementary information 702 may include information which is the same as that of the corresponding information illustrated in FIG. 5, include only information required for the DICOM standard, or include only predetermined items which are arbitrarily set. For example, the subject information 704 may include only information on a subject ID, information on an age, information on a gender, and information on an inspection ID. The supplementary information 702 may not include the probe information 705. Furthermore, the supplementary information 702 may further include timing information corresponding to the timing information 506 of FIG. 5 and operation information corresponding to operation information 1700, although this configuration may not be required since the association information 706 includes operation information and timing information.

According to the configuration of the third embodiment, the information processing apparatus 107 may associate obtained images with each other based on an operation input performed by the user. By this, flicker of photoacoustic images may be reduced when a moving image including the ultrasonic images and the photoacoustic images is displayed in a viewer 113. Furthermore, the viewer 113 may efficiently display a portion associated with an operation input performed by the user based on the association information 706 included in the DICOM object 701. For example, the viewer 113 may easily specify a frame section obtained with photoacoustic image data in a group of frames of continuous ultrasonic images with reference to operation information 1700. Accordingly, a doctor may efficiently make a diagnosis. Specifically, in a case where an instruction for displaying a superposed image obtained by superposing a photoacoustic image on an ultrasonic image is received from the doctor, for example, the viewer 113 reads the time points to1 and to3 included in the operation information 1700 and obtains and displays ultrasonic images and photoacoustic images in a period of time from the time point to1 to the time point to3. By this process, the viewer 113 may reliably display an image desired by the doctor.

### Modification

Although the case where both of an ultrasonic image and a photoacoustic image are stored is described as an example in the foregoing embodiments, the present invention is not limited to this. For example, a storage instruction may be separately input to each of the ultrasonic image and the photoacoustic image.

Although the case where the output controller 304 associates an ultrasonic image with a photoacoustic image is described as an example in the foregoing embodiments, the present invention is not limited to this. For example, the display controller 306 may display an ultrasonic image and a photoacoustic image obtained by the image processor 303 based on timing information. Specifically, the display controller 306 may perform a process for the association described above.

The present invention is also realized by a process of supplying a program which realizes at least one of the functions of the foregoing embodiment to a system or an apparatus through a network or a storage medium and reading and executing the program using at least one processor of a computer included in the system or the apparatus.

Furthermore, the present invention may be realized by a circuit which realizes at least one of the functions (for example, an ASIC).

The information processing apparatuses according to the foregoing embodiments may be realized as a single apparatus or may perform the foregoing processes by combining the plurality of apparatuses with one another in a communication available manner, and both of the cases are embodiments of the present invention. The processes described above may be executed by a common server apparatus or a server group. The information processing apparatus and a plurality of devices included in the information processing system may be at least communicated at a predetermined communication rate and may not be installed in the same facility or the same country.

Embodiments of the present invention include a mode in which programs of software which realizes the functions of the foregoing embodiment are supplied to a system or an apparatus and a computer included in the system or the apparatus reads and executes codes of the supplied programs.

Accordingly, the program codes installed in the computer to realize processes of the embodiments by the computer are also an embodiment of the present invention. Furthermore, an operating system (OS) operated in a computer may perform a portion of an actual process or an entire actual process based on an instruction included in the program read by the computer, and the functions of the embodiments described above may be realized by the process.

Embodiments obtained by appropriately combining the foregoing embodiments may also be included in the present invention.

According to the present invention, photoacoustic images are individually associated with ultrasonic images so that the ultrasonic images and the photoacoustic images are smoothly reproduced as a moving image.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus which obtains an ultrasonic image based on an ultrasonic signal which is associated with a reflection wave of an ultrasonic wave emitted to a subject at a frame rate higher than a frame rate of a photoacoustic image based on a photoacoustic signal which is associated with a photoacoustic wave generated due to emission of light to the subject, the information processing apparatus comprising:
image obtaining means for obtaining a first photoacoustic image based on a first photoacoustic signal obtained at a first time point, a second photoacoustic image based on a second photoacoustic signal obtained at a second time point after the first time point, and a first ultrasonic image based on an ultrasonic signal obtained at a third time point included in a period of time after the first time point and before the second time point; and
processing means for associating at least one of the first and second photoacoustic images with the first ultrasonic image based on the relationship among the first to third time points.

2. The information processing apparatus according to claim 1, wherein the processing means associates one of the photoacoustic images based on the photoacoustic signals which is obtained at one of the first and second time points which is closer to the third time point with the first ultrasonic image.

3. The information processing apparatus according to claim 2,
wherein the image obtaining means further obtains a second ultrasonic image obtained at a fourth time point which is different from the third time point and which is included in the period of time after the first time point and before the second time point, and
the processing means associates the first photoacoustic image with the first and second ultrasonic images when the first time point is closer to the third and fourth time points when compared with the second time point and associates the second photoacoustic image with the first and second ultrasonic images when the second time point is closer to the third and fourth time points when compared with the first time point.

4. The information processing apparatus according to claim 1, further comprising output means for outputting an object including association information regarding association between the ultrasonic images and the photoacoustic images as supplementary information to an external apparatus.

5. The information processing apparatus according to claim 1, further comprising information obtaining means for obtaining timing information including information on the first to third time points.

6. The information processing apparatus according to claim 5, wherein the timing information includes information on timings when the photoacoustic signals are obtained as information on the first and second time points.

7. The information processing apparatus according to claim 5, wherein the timing information includes information on timings when light is emitted to the subject as information on the first and second time points.

8. The information processing apparatus according to claim 5, wherein the timing information includes information on a timing when the ultrasonic signal is obtained as information on the third time point.

9. The information processing apparatus according to claim 5, wherein the timing information includes information on a timing when an ultrasonic wave is transmitted to the subject as information on the third time point.

10. The information processing apparatus according to claim 1, further comprising display control means for displaying at least one of the ultrasonic image and the photoacoustic image obtained by the image obtaining means in a display unit.

11. The information processing apparatus according to claim 10, wherein the display control means displays the photoacoustic image superposed on the ultrasonic image in the display unit.

12. The information processing apparatus according to claim 1, further comprising:
reception means for receiving an instruction for storing the ultrasonic image and the photoacoustic image obtained by the image obtaining means,
wherein the processing means associates one of the photoacoustic images obtained in a period of time indicated by the instruction with each of the ultrasonic images obtained in the period of time.

13. An information processing apparatus comprising:
image obtaining means for obtaining a photoacoustic image based on a photoacoustic signal which is associated with a photoacoustic wave generated due to emission of light to a subject and obtaining an ultrasonic image based on an ultrasonic signal which is associated with a reflection wave of an ultrasonic wave emitted to the subject;
display control means for displaying one of a photoacoustic image obtained at a first time point and a photoacoustic image obtained at a second time point after the first time point which is obtained at a time point closer to a third time point included in a period of time between the first and second time points on an ultrasonic image obtained at the third time point in a superposed manner;
reception means for receiving an instruction for storing the photoacoustic images and the ultrasonic image; and
output means for outputting information for reproducing, using an external apparatus, an image displayed in the display unit in an instructed period of time.

14. An information processing apparatus comprising:
image obtaining means for obtaining a photoacoustic image based on a photoacoustic signal which is associated with a photoacoustic wave generated due to emission of light to a subject and obtaining an ultrasonic image based on an ultrasonic signal which is associated with a reflection wave of an ultrasonic wave emitted to the subject;
processing means for associating at least one of a plurality of photoacoustic images with each of a plurality of ultrasonic images based on information on an operation input performed by a user; and
output means for outputting the ultrasonic images and the photoacoustic images to the external apparatus with association information indicating photoacoustic images associated with the plurality of ultrasonic images and supplemental information including information on an operation input performed by the user.

15. An information processing apparatus which obtains an ultrasonic image based on an ultrasonic signal which is associated with a reflection wave of an ultrasonic wave emitted to a subject at a frame rate higher than a frame rate of a photoacoustic image based on a photoacoustic signal which is associated with a photoacoustic wave generated due to emission of light to the subject, the information processing apparatus comprising:
obtaining means for obtaining a first photoacoustic image based on a photoacoustic signal obtained at a first time point and a plurality of ultrasonic images based on a plurality of ultrasonic signals, at least one of the ultrasonic signals being obtained at a time point different from the first time point; and
processing means for associating the first photoacoustic image with the plurality of ultrasonic images.

16. An information processing method which obtains an ultrasonic image based on an ultrasonic signal which is associated with a reflection wave of an ultrasonic wave emitted to a subject at a frame rate higher than a frame rate of a photoacoustic image based on a photoacoustic signal which is associated with a photoacoustic wave generated due to emission of light to the subject, the information processing method comprising:
obtaining a first photoacoustic image based on a first photoacoustic signal obtained at a first time point, a second photoacoustic image based on a second photoacoustic signal obtained at a second time point after the first time point, and a first ultrasonic image based on an ultrasonic signal obtained at a third time point included in a period of time after the first time point and before the second time point; and
associating at least one of the first and second photoacoustic images with the first ultrasonic image based on the relationship among the first to third time points.

17. A non-transitory storage medium storing a program that causes a computer to execute the information processing method set forth in claim 16.

18. An information processing method comprising:
obtaining a photoacoustic image based on a photoacoustic signal which is associated with a photoacoustic wave generated due to emission of light to a subject;
obtaining an ultrasonic image based on an ultrasonic signal which is associated with a reflection wave of an ultrasonic wave emitted to the subject;
associating at least one of a plurality of photoacoustic images with each of a plurality of ultrasonic images based on information on an operation input performed by a user; and
outputting the ultrasonic images and the photoacoustic images to the external apparatus with association information indicating photoacoustic images associated with the plurality of ultrasonic images and supplemental information including information on an operation input performed by the user.

19. A non-transitory storage medium storing a program that causes a computer to execute the information processing method set forth in claim 18.

20. An information processing method which obtains an ultrasonic image based on an ultrasonic signal which is associated with a reflection wave of an ultrasonic wave emitted to a subject at a frame rate higher than a frame rate of a photoacoustic image based on a photoacoustic signal which is associated with a photoacoustic wave generated due to emission of light to the subject, the information processing method comprising:
obtaining a first photoacoustic image based on a photoacoustic signal obtained at a first time point and a plurality of ultrasonic images based on a plurality of ultrasonic signals, at least one of the ultrasonic signals being obtained at a time point different from the first time point; and
associating the first photoacoustic image with the plurality of ultrasonic images.

21. A non-transitory storage medium storing a program that causes a computer to execute the information processing method set forth in claim 20.
